(19) United States
(12) Patent Application Publication
(10) Pub. No.: US 2021/0038651 A1
(43) Pub. Date: Feb. 11, 2021

(54) IPSC-DERIVED CELL COMPOSITIONS, AND RELATED SYSTEMS AND METHODS FOR CARTILAGE REPAIR

(71) Applicant: Orig3n, Inc., Boston, MA (US)

(72) Inventors: Robin Y. Smith, Boston, MA (US); Marcie A. Glicksman, Boston, MA (US); Lizbeth Cornivelli, Boston, MA (US)

(21) Appl. No.: 16/978,362

(22) PCT Filed: Mar. 5, 2019
(86) PCT No.: PCT/US19/20804

§ 371 Date: Sep. 4, 2020

Related U.S. Application Data
(60) Provisional application No. 62/733,526, filed on Sep. 19, 2018.
(60) Provisional application No. 62/730,454, filed on Sep. 12, 2018.
(60) Provisional application No. 62/700,210, filed on Jul. 18, 2018.
(60) Provisional application No. 62/639,442, filed on Mar. 6, 2018.

(51) Int. Cl. A61K 35/28; A61P 19/00; A61K 9/00; A61K 35/32; C12N 5/077

(52) U.S. Cl. CPC A61K 35/28; A61P 19/00; C12N 5/0655; A61K 35/32; A61K 9/0024

(57) ABSTRACT

Presented herein are personalized compositions comprising iPSCs and/or iPSC-derived cells (cells) and methods of producing personalized compositions suitable for various therapies, including chondrogenesis therapies, to be administered to an individual or a group of individuals. The cells and/or cell lines, and any compositions derived therefrom, are identified as compatible with a specific individual or specific group of individuals using an identification of a cell type indicative of compatibility such as an HLA match. The compatible cells are then used to derive “personalized” compositions, wherein the “personalized” compositions comprise one or more cell-secreted molecules suitable for therapy. It is found herein that a composition comprising one or more iPSC-derived MSCs, iPSC-derived chondrocytes, and iPSC-derived chondrons may provide improved treatment efficacy than would be offered by bone marrow-MSCs (BM-MSCs) or compositions comprising BM-MSCs.

### CROSS REFERENCE TO RELATED APPLICATIONS

This application claims the benefit of U.S. Provisional Application No. 62/639,442 filed Mar. 6, 2018, U.S. Provisional Application No. 62/700,210 filed Jul. 18, 2018, U.S. Provisional Application No. 62/730,454 filed Sep. 12, 2018, and U.S. Provisional Application No. 62/733,526 filed Sep. 19, 2018, the contents of which are hereby incorporated by reference herein in their entirety.

### TECHNICAL FIELD

The invention relates generally to compositions derived from induced pluripotent stem cells (iPSCs) and/or iPSC-derived cells, and related systems and methods.

### BACKGROUND

Chondrocytes are cells found in cartilage. They function to make and maintain the cartilage. Chondrocytes are found embedded in extracellular matrix and produce all of the structural components of cartilage, including collagen, proteoglycans, glycosaminoglycans, and glycoproteins. A chondrocyte and the surrounding pericellular matrix (PCM) together constitute a chondron, considered the primary structural, functional, and metabolic unit of cartilage. These cells are found in healthy cartilage. In normal adult articular cartilage, chondrocytes are stable, post-mitotic, differentiated cells that maintain tissue homeostasis by synthesizing very low levels of extracellular matrix (ECM) components to replace damaged molecules, thus preserving the structural integrity of the cartilage matrix. They produce and maintain the cartilaginous ECM, which comprises collagen, proteoglycans, and glycosaminoglycans. The chondrocytes are not only responsible for the synthesis of collagen proteoglycans, and glycosaminoglycans, but also enzymes that degrade matrix components (collagenase, neutral proteinases, and cathepsins). This dual function places the chondrocyte in the role of regulating cartilage synthesis and degradation.

Chondrocytes originate from Mesenchymal stem cells (MSCs), which are undifferentiated stem cells from the mesoderm. These MSCs can be differentiated into a variety of generative cells including bone cells (osteoblasts), fat cells (adipocytes), and cartilage cells (chondrocytes). During cell differentiation to form chondrocytes, the undifferentiated MSCs lose their multipotency, multiply and cluster together in a dense aggregate of chondrogenic cells, or cartilage, at the location of chondrification. Next, these chondrogenic cells differentiate into chondroblasts, which then differentiate further into chondrocytes that synthesize the extra-cellular cartilaginous matrix.

Cartilage provides the basis for the developing skeleton and thus mutations in genes for cartilage-specific proteins often produce developmental abnormalities. Osteoarthritis, a widespread disease of diarthrodial joints, can be caused by injuries to the knee cartilage including lesions disrupting both cartilage and subchondral bone (osteochondral lesions), and lesions limited to the cartilage tissue (chondral lesions). Further, chondrocytes do not compensate for matrix damage induced externally. For example, these external factors include, but are not limited to mechanical stress or enzymatic degradation through synovial proteases.

The usual treatment for severe osteoarthritis is replacement of the arthritic articular surface with an artificial prosthesis. While total knee replacement is commonly performed in patients over 60 years of age, treatment of younger patients is difficult as the artificial prosthesis has a limited lifetime. Thus, transplanted chondrocytes are potential treatments for focal articular cartilage defects. These transplants may be autologous (obtained from the same individual (e.g., the patient)) or allogeneic (derived from separate individuals of the same species).

Where allogeneic cells are needed, a suitable donor (someone other than the patient) must be found for the patient in order to minimize risk of rejection and maximize chances for success. Donor registries are services that seek to match registered donors with patients in need of an allogeneic transplant. Matching based on human leukocyte antigen (HLA) typing is performed to find suitable donors. Because there are many different HLA types, it is often difficult to find suitable matches, particularly when no family members of the patient are an HLA-identical match. The term “super donors” refers to human leukocyte antigen (HLA) types (or cell lines or individuals having those HLA types) that do not trigger strong rejection reactions. Human cells have two sets of HLA markers, one from each parent. An individual who inherits two identical copies (called homozygous) of these HLA markers are considered “super donors”. Similar to the way someone with type A blood can donate to people with either A or AB blood types, a super donor's cells can serve a greater portion of the population.

Humans are almost always heterozygous for a particular HLA gene—that is, genotyping data shows that humans usually express two different alleles. For a successful match, eight (8) HLA alleles are best for matching (4 alleles on each of the donor and recipients chromosomes). With homozygous donors, only 4 alleles are required to be matched, therefore increasing the number of recipients that would be a match to the donor. Individuals that are homozygous for at least three key HLA alleles , HLA-A, HLA-B, and HLA-DRB, that govern rejection means that only three genes need to be genotyped and matched instead of six genes. iPSCs can be differentiated into a variety of different cell types.

iPSC lines derived from so-called “super donors” can be used to reduce immunogenicity of differentiated cells upon transplantation. It is believed that about 200 such iPSC lines could cover a high percentage (e.g., at least 90%, at least 95%, or more) of the U.S. and/or European population, and about 90 to 100 such iPSC lines could cover a high percentage (e.g., at least 90%, at least 95%, or more) of the Japanese population.

Autologous chondrocyte implantation has been demonstrated as a treatment of various cartilage defects, disorders, conditions, and diseases. See, for example, “Matrix-Induced Autologous Chondrocyte Implantation Versus Microfracture In The Treatment Of Cartilage Defects Of The Knee: A 2-Year Randomised Study”, Knee Surgery, Sports Traumatology, Arthroscopy, 2010 Jan; 18(4): 519-527; “Treatment of Deep Cartilage Defects in the Knee with Autologous Chondrocyte Transplantation”, The New England Journal of Medicine, 1994 Oct; 331: 889-895; “Treatment Of Osteochondritis Dissecans Of The Knee With Autologous Chondrocyte Transplantation: Results At Two To Ten Years”, The Journal of Bone and Joint Surgery, 2003; 85: 17-24; “A Prospective Study of Autologous Chondrocyte Implantation in Patients with Failed Prior Treatment for Articular Cartilage Defect of the Knee”, The American Journal of Sports Medicine, 2017 Aug; 37(1): 42-55; “Early postoperative adherence of matrix-induced autologous chondrocyte implantation for the treatment of full-thickness cartilage defects of the femoral condyle”, Knee Surgery, Sports Traumatology, Arthroscopy, 2005 Sept; 13(6):451-457; “Treatment Outcomes of Autologous Chondrocyte Implantation for Full-Thickness Articular Cartilage Defects of the Trochlea”, The American Journal of Sports Medicine, 2007 Jun; 35(6):915-921; and “Generation of pluripotent stem cells and their differentiation to the chondrocytic phenotype”, Methods Mol. Med., 2004; 100:53-68; the contents of each of which are incorporated herein by reference.

Furthermore, in recent years, there have been significant advances in the use of allogeneic treatments to repair cartilage defects, conditions, and diseases. For example, allogeneic chondrocyte transplants into rabbits and hens were made by grafting chondrocytes cultivated in artificial scaffolds and thus protected by the matrix produced in vitro. See, for example, K. Moskalewski, A. Hyc, and A. Osiecka-Iwan, “Immune response by host after allogeneic chondrocyte transplant to the cartilage,” Microscopy Research and Technique, 2002 Jul; 58(1): 3-13. In other research, chondrocytes from juvenile cadaveric donors were studied as an alternative to autologous cells, which shows allogeneic juvenile chondrocytes do not stimulate an immunologic response in vivo. See, for example, H. D. Adkisson, J. A. Martin, R. L. Amendola et al., “The Potential of Human Allogeneic Juvenile Chondrocytes for Restoration of Articular Cartilage,” The American Journal of Sports Medicine, 2010 Apr; 38(7) 1324-1333; the contents of each of which are incorporate herein by reference.

Every cell, including chondrocytes, has a unique transcriptome. The transcriptome generally refers to the totality of RNA molecules (RNA transcripts) expressed from genes in a cell, or group of cells, or an organism. The term transcriptome has been variously applied to the total set of transcripts in a given organism, or to a specific subset of transcripts in a particular cell or cell type (e.g., mRNA, tRNA, sRNA, miRNA). Since the transcriptome refers to the transcripts or actively expressed genes at a given point in time in a cell, cell type, or organism, the transcriptome can vary with external factors like environmental conditions.

Transcriptomics technologies are the techniques used to measure and study the transcriptome of an organism or cell. There are two major techniques used in this field: i) microarrays, and ii) next generation sequencing (RNA-Seq). While microarrays measure a set of predetermined sequences, RNA-Seq uses high-throughput sequencing to sequence all the transcripts. Measuring the expression genes through measurement of the transcripts in an organism or a cell at a given instant in time or under various external stimuli provides information about how genes are regulated and can enhance an understanding of the biology of the organism or cell. Such knowledge can provide insight into the functions of previously unannotated genes, the understanding of human disease, and general broad coordinated trends that were otherwise difficult to discern. Transcriptomes of autologous cells or allogeneic cells may be studied using the aforementioned technologies.

Recently, transcriptomes have been successfully produced from iPSCs and also used for treatment of various cosmetic conditions and diseases. See, for example, “Exosomes Generated From iPSC-Derivatives New Direction for Stem Cell Therapy in Human Heart Diseases”, Cir. Res. 2017 Jan; 120(2): 407-417; “The secretome of induced pluripotent stem cells reduces lung fibrosis in part by hepatocyte growth factor”, Stem Cell Res. Ther. 2014 Nov; 5(123): 1-11; “Exosomes secreted by human-induced pluripotent stem cell-derived mesenchymal stem cells attenuate limb ischemia by promoting angiogenesis in mice”, Stem Cell Res. Ther. 2015 Apr; 6(10): 1-15; “Induced pluripotent stem cell (iPSCs) and their application in immunotherapy”, Cell Mol. Immunol. 2014 Jan; 11(1): 17-24; “Human growth factor and cytokine skin cream for facial skin rejuvenation as assessed by 3D in vivo optical skin imaging”, J. Drugs Dermatol. 2007 Oct; 6(10): 1018-23; “Skin rejuvenation using cosmetic products containing growth factors, cytokines, and matrikines: a review of the literature,” J. Drugs Dermatol., 2007 Feb; 6(2): 197-200; and “Anti-cytokine therapy for Rheumatoid Arthritis,” Blood, 2000 Feb; 51: 207-29; the contents of each of which are incorporated herein by reference. Furthermore, in recent years, there have been significant advances in the production of iPSCs from cells collected from a biological sample of a subject (e.g., blood cells). For example, iPSCs can be made by inserting copies of stem cell-associated genes—e.g., Oct 3/4, Sox 2, Klf4, and c-Myc (or Oct 3/4, Sox 2, Nanog, and Lin28)—into cells collected from the biological sample using viral vectors. See, for example, K. Okita, T. Ichisaka, and S. Yamanaka, “Generation of germline-competent induced pluripotent stem cells,” Nature, vol. 448, no. 7151, pp. 313-317, 2007; K. Okita, Y. Matsumura, Y. Sato et al., “A more efficient method to generate integration-free human iPS cells,” Nature Methods, vol. 8, no. 5, pp. 409-412, 2011; the contents of each of which are incorporate herein by reference.

There is a need for more effective long-term storage and utilization of chondrogenic compositions and advances in methods of producing them. Furthermore, there is a need for more effective chondrocyte therapy, e.g., for cartilage repair.

### SUMMARY

Presented herein are personalized induced pluripotent stem cells (iPSC)-derived compositions and methods of producing personalized iPSC-derived compositions suitable for chondrocyte based therapy (e.g., suitable for chondrocyte therapy for cartilage repair), or suitable for other therapies, to be administered to a specific individual and/or a specific group of individuals. In certain embodiments, reserves of biological material are stored in a managed physical repository (e.g., a bank) for providing a resource (e.g., donors for chondrocyte therapy for cartilage repair) for patients. In certain embodiments, these reserves include induced pluripotent stem cells (iPSCs) and other iPSC-derived cells [e.g., hematopoietic stem cells (HSCs), blood progenitor cells, Retinal Pigment Epithelium (RPE), chondrocytes, mesenchymal stem cells (MSCs), embryoid bodies and the like], iPSC lines and other iPSC-derived cell lines [e.g., HSC lines, blood progenitor cell lines, MSC lines, RPE lines, and the like], as well as chondrocytes derived from these cells and/or cell lines, This managed repository of cells, and/or cell lines, and/or compositions derived from iPSCs (or embryoid bodies formed from iPSCs), has associated with it corresponding data comprising a set of characterized HLA loci, said corresponding data being stored in a searchable database for retrieval of one or more matching physical cell lines upon query, said database being either co-located or remotely located in relation to the physical repository. The physical repository comprises a bank of cells (e.g., iPSCs, embryoid bodies, HSCs, MSCs, RPEs, blood progenitor cells and/or various other cells) derived from iPSCs, cell lines (HSCs, MSCs, RPEs, blood progenitor cells and/or various other cell lines derived from iPSCs), along with compositions derived from each of these cells and/or cell lines (e.g., iPSC-derived chondrogenic compositions), for each of a set of HLA types. This repository of cells, and/or cell lines and/or iPSC-derived compositions allows for identification and provision of allogenic cell lines and iPSC-derived compositions (e.g., iPSC-derived chondrogenic compositions) suitable for transplantation and/or treatment to reestablish normal function (e.g., cartilage function) in patients with various diseases and/or conditions. In certain embodiments, iPSC-derived chondrogenic compositions may comprise one or more of (i)-(iii) as follows: (i) Induced Pluripotent Stem Cell (iPSC)-derived Mesenchymal Stem Cells (MSCs), (ii) iPSC-derived chondrocytes, and (iii) iPSC-derived chondrons. Such iPSC-derived chondrogenic compositions, as disclosed herein, may be typed and/or characterized.

It is found herein that there are significant transcriptome differences between human bone marrow MSCs (BM-MSC) and the herein-created iPSC-derived MSCs (ORIG3N-MSCs) according to multiplex transcriptome analysis (AmpliSeq). It is found that the number of transcripts of genes relevant to chondrogenesis, inflammatory pathways, and collagens are generally higher in the iPSC-derived MSCs than the BM-MSCs. This indicates that a composition created from iPSC-derived MSCs may provide improved performance in a therapy, e.g., cartilage and/or bone therapy (e.g., cartilage therapy by direct administration of the composition in a joint), than would be offered by BM-MSCs or compositions made therefrom. Furthermore, iPSC-derived MSCs are more homogeneous than MSCs derived from bone marrow or other tissue (e.g., adipocytes, fat).

In certain embodiments, therapy is allogeneic, though autologous therapy is also contemplated herein, in other embodiments. In certain embodiments, the chondrocyte therapy composition comprises iPSC-derived MSCs and/or iPSC-derived chondrocytes (e.g., iPSC-derived chondrocytes may be chondrocytes derived from MSCs that were, themselves, derived from iPSCs). In certain embodiments, the chondrocyte therapy composition comprises a transcriptome, or portion thereof, of iPSC-derived MSCs. In certain embodiments, the composition comprises a transcriptome, or portion thereof, of iPSC-derived chondrocytes. In certain embodiments, the composition comprises a secretome, or portion thereof, of (i) iPSC-derived MSCs and/or (ii) iPSC-derived chondrocytes and/or (iii) iPSC-derived chondrons. In certain embodiments, the composition comprises any one or more of (i) to (vi) as follows: (i) iPSC-derived MSCs; (ii) iPSC-derived chondrocytes; (iii) a transcriptome, or portion thereof, of iPSC-derived MSCs; (iv) a transcriptome, or portion thereof, of iPSC-derived chondrocytes; (v) a secretome, or portion thereof, of iPSC-derived MSCs; (vi) a secretome, or portion thereof, of iPSC-derived chondrocytes; and (vii) iPSC-derived chondrons.

In certain embodiments, treatment comprises administration (e.g., injection) of the chondrocyte therapy composition in one or more joints of a subject (e.g., to promote cartilage growth therein).

In certain embodiments, reserves of biological material are stored in a managed physical repository (e.g., a bank) for providing a resource (e.g., donors for chondrocyte therapy for cartilage repair) for patients. In certain embodiments, these reserves include the heretofore mentioned compositions. In certain embodiments, the reserves alternatively or additionally include induced pluripotent stem cells (iPSCs) and/or other iPSC-derived cells [e.g., hematopoietic stem cells (HSCs), blood progenitor cells, Retinal Pigment Epithelium (RPE), chondrocytes, mesenchymal stem cells (MSCs), embryoid bodies and the like], iPSC lines and other iPSC-derived cell lines [e.g., HSC lines, blood progenitor cell lines, MSC lines, REP lines, and the like], e.g., from which the chondrocyte therapy compositions are made. This managed repository of cells, and/or cell lines, and/or chondrocyte therapy compositions derived from iPSCs (or embryoid bodies formed from iPSCs), has associated with it corresponding data comprising a set of characterized HLA loci, said corresponding data being stored in a searchable database for retrieval of one or more matching physical cell lines upon query, said database being either co-located or remotely located in relation to the physical repository. The physical repository comprises a bank of cells (e.g., iPSCs, embryoid bodies, HSCs, MSCs, RPEs, blood progenitor cells and/or various other cells) derived from iPSCs, cell lines (HSCs, MSCs, RPEs, blood progenitor cells and/or various other cell lines derived from iPSCs), along with chondrocyte therapy compositions derived from each of these cells and/or cell lines (E.g., iPSC-derived chondrogenic compositions), for each of a set of HLA types (e.g., including identification of “super donors”). This repository of cells, and/or cell lines and/or iPSC-derived chondrocyte therapy compositions allows for identification and provision of allogeneic cell lines and iPSC-derived compositions suitable for transplantation and/or treatment to reestablish normal cartilage function in patients with various diseases and/or conditions.

In certain embodiments, iPSCs and/or MSCs from which the chondrocyte therapy composition is derived and/or made are genetically modified, e.g., via ZFN (Zinc-finger nucleases), TALEN (Transcription activator-like effector nucleases), CRISPR-Cas9 (Clustered Regularly Interspaced Short Palindromic Repeats, CRISPR-associated protein-9 nuclease), and/or other genome editing technology, prior to (or as part of) manufacture of the chondrogenic composition in order to further improve or optimize the therapy for its intended purpose (e.g., cartilage repair). For example, in certain embodiments, iPSCs, or cells differentiated from iPSCs, are engineered using one or more various technologies (e.g., CRISPR/Cas9) to upregulate production of one or more desired proteoglycans or glycosaminoglycans in the chondrogenic composition. For example, in certain embodiments, an iPS cell (and/or cell(s) derived therefrom) may be genetically modified (e.g., via CRISPR-Cas9 genome editing and/or gene transfer) to remove, replace, and/or edit one or more genes to result in (or to increase the likelihood of) the upregulation of one or more desired proteoglycans or glycosaminoglycans in the iPSC-derived chondrocyte therapy composition. In certain embodiments, an iPSC cell (and/or cell(s) derived therefrom) may be genetically modified to result in (or increase the likelihood of) the downregulation of certain cellular components.

Furthermore, the techniques described herein allow for the tuning of treatment compositions to a specific individual or a specific group of individuals, thus enabling improved methods of chondrocyte based therapy, e.g. due to an enhanced compatibility of the specific individual or group of individuals with the cells from which the desired chondrocyte therapy composition is derived. Also, allogeneic iPS cells and/or cell lines that are compatible with a large portion of a specific population, e.g. super donors, can be prepared and stored in advance for large groups of individuals. These super donor-derived chondrocyte therapy compositions can then be made immediately available to people who need them, thus reducing production times of the iPSC-derived compositions.

In one aspect, the invention is directed to a method of treatment comprising administering a composition to a subject in need thereof, wherein the composition comprises one or more of (i)-(iii) as follows: (i) iPSC-derived Mesenchymal Stem Cells (MSCs), (ii) iPSC-derived chondrocytes, and (iii) iPSC-derived chondrons, wherein the iPSCs from which the one or more of (i)-(iii) were derived have been characterized by HLA typing to determine compatibility for administration to the subject.

In certain embodiments, the one or more of (i) iPSC-derived Mesenchymal Stem Cells (MSCs), (ii) iPSC-derived chondrocytes, and (iii) iPSC-derived chondrons was retrieved from an indexed-biorepository.

In certain embodiments, the composition comprises iPSC-derived MSCs. In certain embodiments, the iPSC-derived MSCs have a transcriptome that comprises transcripts of one or more genes (e.g., at least one, at least two, at least three, at least five, at least seven, at least ten genes) selected from the group consisting of CXCR4, CXCR7, CCL5 (RANTES), IDO1, A2M, EGFL6, BMP2, BMP4, BMPR1B, IGF2, CILP2, COL2A1.

In certain embodiments, the treatment comprises reduction of inflammation.

In certain embodiments, the treatment comprises repair of cartilage.

In certain embodiments, the composition comprises iPSC-derived MSCs and iPSC-derived chondrocytes (e.g., iPSC-derived chondrocytes in the form of chondrons, and/or compositions of chondrocytes prepared from iPSC-derived chondrons, e.g., prepared by thawing frozen iPSC-derived chondrons). In certain embodiments, a ratio of iPSC-derived MSCs to iPSC-derived chondrocytes is from approximately 0.1:1 to approximately 1:1. In certain embodiments, a ratio of iPSC-derived chondrocytes to iPSC-derived MSCs is from approximately 0.1:1 to approximately 1:1.

In certain embodiments, the composition comprises iPSC-derived chondrons.

In certain embodiments, the administering step comprises administering the composition by injection.

In certain embodiments, the administering step comprises administering the composition by implantation.

In certain embodiments, the composition is frozen prior to the administering step.

In certain embodiments, the composition is thawed prior to the administering step.

In certain embodiments, the method comprises storing the composition in an indexed-biorepository prior to the administering step.

In certain embodiments, the composition was retrieved from an indexed-biorepository prior to the administering step.

In certain embodiments, the subject is suffering from a disease, a disorder, or an injury that causes cartilage loss and/or damage.

In certain embodiments, the administering step comprises administering a unit dose of at least approximately 150k iPSC-derived cells.

In certain embodiments, the composition is a chondrogenic solution comprising chondrocytes at a concentration of 3 million cells per mL or greater. In some embodiments, the chondrogenic solution comprise chondrocytes dissociated from chondrons, wherein the chondrocytes are at a concentration of 3 million cells per mL or greater.

In certain embodiments, the composition comprises iPSC-derived MSCs and iPSC-derived chondrocytes (e.g., iPSC-derived chondrocytes in the form of chondrons, and/or compositions of chondrocytes prepared from iPSC-derived chondrons, e.g., prepared by thawing frozen iPSC-derived chondrons), wherein the ratio of iPSC-derived MSCs to iPSC-derived chondrocytes in the composition is approximately 1:1.

In another aspect, the invention is directed to use of a composition for the manufacture of a medicament for treatment of a disease, disorder, or condition, wherein the treatment comprises administration of the medicament to a subject in need thereof, wherein the composition comprises one or more of (i)-(iii) as follows: (i) iPSC-derived Mesenchymal Stem Cells (MSCs), (ii) iPSC-derived chondrocytes, and (iii) iPSC-derived chondrons, wherein the iPSCs have been characterized by HLA typing to determine compatibility for administration to the subject. In some embodiments, the condition in the subject is a disease, disorder, or injury that causes cartilage damage and/or cartilage loss, and/or inflammation.

In another aspect, the invention is directed to of a composition in the manufacture of a medicament for treatment of a cartilage injury, damage, or defect, wherein the treatment comprises administration of the medicament to a subject in need thereof, wherein the composition comprises one or more of (i)-(iii) as follows: (i) iPSC-derived Mesenchymal Stem Cells (MSCs), (ii) iPSC-derived chondrocytes, and (iii) iPSC-derived chondrons, wherein the iPSCs have been characterized by HLA typing to determine compatibility for administration to the subject.

In another aspect, the invention is directed to a composition comprising one or more of (i)-(iii) as follows: (i) iPSC-derived Mesenchymal Stem Cells (MSCs), (ii) iPSC-derived chondrocytes, and (iii) iPSC-derived chondrons, for use in a method of treating damage or injury to cartilage, wherein the iPSCs have been characterized by HLA typing to determine compatibility for treatment of the subject.

In another aspect, the invention is directed to a composition comprising one or more of (i)-(iii) as follows: (i) iPSC-derived Mesenchymal Stem Cells (MSCs), (ii) iPSC-derived chondrocytes, and (iii) iPSC-derived chondrons, for use in a therapy, wherein the iPSCs have been characterized by HLA typing to determine compatibility for therapeutic administration to a subject.

In another aspect, the invention is directed to a composition comprising one or more of (i)-(iii) as follows: (i) Induced Pluripotent Stem Cell (iPSC)-derived Mesenchymal Stem Cells (MSCs), (ii) iPSC-derived chondrocytes, and (iii) iPSC-derived chondrons, wherein the iPSCs have been characterized by HLA typing.

In certain embodiments, the composition is injectable.

In certain embodiments, the composition is implantable.

In certain embodiments, the composition is frozen.

In certain embodiments, the composition is thawed.

In certain embodiments, the composition is stored in an indexed-biorepository.

In certain embodiments, the composition is retrieved from an indexed-biorepository.

In certain embodiments, the one or more of (i) iPSC-derived Mesenchymal Stem Cells (MSCs), (ii) iPSC-derived chondrocytes, and (iii) iPSC-derived chondrons are retrieved from an indexed biorepository.

In certain embodiments, the composition comprises iPSC-derived MSCs. In certain embodiments, the iPSC-derived MSCs have a transcriptome that comprises transcripts of one or more genes (e.g., at least one, at least two, at least three, at least five, at least seven, at least ten genes) selected from the group consisting of CXCR4, CXCR7, CCL5 (RANTES), IDO1, A2M, EGFL6, BMP2, BMP4, BMPR1B, IGF2, CILP2, COL2A1.

In certain embodiments, the number of transcripts of each of one or more genes indicated in Table 2 of the iPSC-derived MSCs is equal to or greater than the corresponding number of transcripts of bone marrow-derived MSCs (BM-MSCs) of the same HLA type.

In certain embodiments, the composition is administered as treatment to a subject suffering from a disease, a disorder, or an injury that causes cartilage loss and/or damage.

In certain embodiments, the unit dose comprises at least approximately 150k iPSC-derived cells.

In certain embodiments, the composition is a chondrogenic solution comprising chondrons at a concentration of 3 million cells per mL or greater. In some embodiments, the chondrogenic solution comprises chondrocytes dissociated from chondrons, wherein the chondrocytes are at a concentration of 3 million cells per mL or greater.

In certain embodiments, the composition comprises iPSC-derived MSCs and iPSC-derived chondrocytes (e.g., iPSC-derived chondrocytes in the form of chondrons, and/or compositions of chondrocytes prepared from iPSC-derived chondrons, e.g., prepared by thawing frozen iPSC-derived chondrons). In certain embodiments, a ratio of iPSC-derived MSCs to iPSC-derived chondrocytes in the composition is from approximately 0.1:1 to approximately 1:1. In certain embodiments, a ratio of iPSC-derived chondrocytes to iPSC-derived MSCs in the composition is from approximately 0.1:1 to approximately 1:1. In certain embodiments, a ratio of iPSC-derived MSCs to iPSC-derived chondrocytes in the composition is approximately 1:1.

In certain embodiments, the composition comprises iPSC-derived chondrons.

In another aspect, the invention is directed to a method of manufacturing a composition comprising one or more of (I)-(III) as follows: (I) iPSC-derived Mesenchymal Stem Cells (MSCs), (II) iPSC-derived chondrocytes, and (III) iPSC-derived chondrons tailored for treatment of a subject, said method comprising the steps of: (a) identifying, as compatible with the subject, one or both of (i) and (ii) as follows: (i) one or more induced pluripotent stem (iPS) cells and/or iPSC-derived cells, said cells being of one or more HLA types each of which is compatible with the subject, and (ii) one or more iPS cell lines and/or one or more iPSC-derived cell lines, said cell lines being of one or more HLA types each of which is compatible with the subject; (b) retrieving compatible cells corresponding to the one or more cells and/or cell lines identified as compatible with the subject; and (c) producing the composition using the retrieved compatible cells.

In certain embodiments, the compatible cells and/or cells lines are human cells and/or human cell lines.

In certain embodiments, the compatible cells and/or cells lines are non-human animal cells and/or non-human animal lines cells.

In certain embodiments, the compatible cells and/or cell lines are derived from the subject.

In certain embodiments, the compatible cells and/or cell lines are derived from an individual other than the subject.

In certain embodiments, the composition comprises iPSC-derived chondrocytes and one or more compatible-cell-secreted species suitable for cartilage repair of the subject, wherein the compatible cell-secreted species are one or more members selected from the group consisting of collagen, proteoglycans, glycosaminoglycans, exosomes, and microvesicles.

In certain embodiments, the composition comprises iPSC-derived chondrons.

In certain embodiments, step (c) comprises producing a macroscopic cartilage structure from the retrieved compatible cells or from chondrocytes derived from the retrieved compatible cells.

In certain embodiments, step (c) comprises 3D-printing a macroscopic cartilage structure using the composition, wherein the composition is produced from the retrieved compatible cells and/or from chondrocytes derived from the retrieved compatible cells.

In certain embodiments, step (c) comprises extracting one or more cell-secreted species from the retrieved compatible cells, wherein the cell-secreted species are one or more members selected from the group consisting of collagen, proteoglycans, glycosaminoglycans, exosomes, and microvesicles.

In certain embodiments, step (b) comprises deriving the compatible cells from a biological sample of the subject.

In certain embodiments, the method further comprises (d) freezing the iPSC-derived iPSC-derived Mesenchymal Stem Cells (MSCs), (II) iPSC-derived chondrocytes, and/or (III) iPSC-derived chondrons.

In certain embodiments, the retrieved compatible cells comprise one or more members selected from the group consisting of iPSCs, MSCs, Retinal Pigment Epithelium (RPEs), chondrocytes, hematopoietic stem cells (HSCs), blood progenitor cells, embryoid bodies, and other iPSC-derived cells.

In certain embodiments, the subject is human.

In certain embodiments, step (b) comprises obtaining the compatible cells from a physical repository.

In certain embodiments, step (b) comprises retrieving the compatible cells using a processor-based query from a user, wherein the query comprises an identification of a cell type indicative of compatibility with the subject. In certain embodiments, the identification of cell type indicative of compatibility with the subject comprises an HLA match.

In certain embodiments, the composition comprises the retrieved compatible cells.

In certain embodiments, producing the composition in step (c) comprises exposing the compatible cells to culture and/or differentiation media. In certain embodiments, the composition comprises the compatible cells, the culture media, the differentiation media, and one or more compatible-cell-secreted species, wherein the compatible cell-secreted species are one or more members selected from the group consisting of collagen, proteoglycans, glycosaminoglycans, exosomes, and microvesicles.

In certain embodiments, the method comprises dedifferentiating and/or differentiating the one or more iPS-derived cells and/or cell lines identified as compatible with the subject to produce mesenchymal stem cells (MSCs) and/or chondrocytes. In certain embodiments, the method comprises producing the composition from the MSCs and/or chondrocytes.

In certain embodiments, the composition is a treatment spray.

In certain embodiments, the composition is applied topically.

In certain embodiments, the composition is a treatment injection.

In certain embodiments, step (b) comprises obtaining the compatible cells from a physical repository, wherein the physical repository comprises an iPS cell line derived from the subject, and wherein step (b) comprises: storing, by a processor of a computing device, a database comprising a data entry corresponding to each of the iPS cell lines in the physical repository; receiving, by the processor, a query from a user comprising an identification of the subject; and matching, by the processor, the query to a data entry of the database, thereby identifying as compatible with the subject the iPS cell line derived from the subject.

In certain embodiments, step (b) comprises: storing, by a processor of a computing device, a database comprising a data entry corresponding to each of a plurality of characterized iPS cell and/or iPS cell lines and/or iPSC-derived cell and/or iPSC-derived cell lines or corresponding embryoid bodies, the data entry for each iPS cell and/or iPS cell line and/or iPSC-derived cell and/or iPSC-derived cell line comprising a set of characterized HLA loci corresponding to the iPS cell and/or iPS cell line and/or iPSC-derived cell and/or iPSC-derived cell line; receiving, by the processor, a query from a user, the query comprising a set of queried HLA loci for the subject; and retrieving, by the processor, one or more data entries of the database, each representative of an iPS cell and/or cell line and/or an embryoid body and/or an HSC line and/or a blood progenitor line and/or MSC line and/or RPE line and/or chondrocyte line derived from an iPS cell and/or cell line matching the queried HLA loci, thereby identifying cells that match the queried HLA loci for the subject as compatible with the subject.

In certain embodiments, the retrieved data entries of the database are exactly matching, partially matching, and/or are identified as compatible with the queried HLA loci. In certain embodiments, the set of characterized HLA loci comprises at least 3 given loci, wherein the given loci are HLA-A, HLA-B, and HLA-DRB. In certain embodiments, the set of characterized HLA loci comprises at least 9 given loci, wherein the given loci are HLA-A, HLA-B, HLA-C, HLA-DRB1, HLA-DRB3, HLA-DRB4, HLA-DRB5, HLA-DQB1, and HLA-DPB1. In certain embodiments, the set of characterized HLA loci comprises at least 3 given loci, wherein the given loci are members selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the method further comprises retrieving characterized cells from the physical repository according to the one or more retrieved data entries matching the queried HLA loci. In certain embodiments, the retrieved characterized cells are one or more members selected from the group consisting of iPS cells, iPS cell lines, embryoid bodies, blood progenitor cells, HSCs, MSCs, RPEs, chondrocytes, iPSC-derived cells, and iPSC-derived cell lines. In certain embodiments, the method further comprises producing MSCs and/or chondrocytes from iPSCs and/or embryoid bodies and/or HSCs and/or blood progenitor cells and/or RPEs of an iPSC line corresponding to the one or more retrieved data entries matching the queried HLA loci. In certain embodiments, the database comprises a data entry corresponding to each of a plurality of iPS super donor cell lines, the data entry for each super donor cell line comprising a set of characterized HLA loci corresponding to the super donor cell line. In certain embodiments, each of the plurality of iPS super donor cell lines can be used for treatment of a particular subject or particular group of subjects having matching HLA loci with lower risk of immune rejection by the particular subject or particular group of subjects.

In certain embodiments, the method further comprises administering the composition to the subject. In certain embodiments, the administering step comprises administering the composition to the subject for treatment of a known disease, injury, or condition in the subject, wherein the known disease, injury, or condition is a member selected from the group consisting of rheumatic diseases, cancer, cartilage damage, chondropathy, relapsing polychondritis, osteochondritis dissecans, costochondritis, Chondromalacia patellae, arthritis, and inflammation.

In another aspect, the invention is directed to a treatment comprising a therapeutically effective amount of a composition comprising one or more of (I)-(III) as follows: (I) Induced Pluripotent Stem Cell (iPSC)-derived Mesenchymal Stem Cells (MSCs), (II) iPSC-derived chondrocytes, and (III) iPSC-derived chondrons, for use in a method of treating cartilage loss and/or damage in a subject, wherein the composition is manufactured using one or both of (i) and (ii) as follows: (i) one or more induced pluripotent step (iPS) cells and/or iPSC-derived cells identified as compatible with the subject; and (ii) one or more iPS cell lines and/or one or more iPSC-derived cell lines, wherein the cells and/or cell lines are of one or more HLA types identified as compatible with the subject.

In certain embodiments, the compatible cells and/or cells lines are human cells and/or human cell lines.

In certain embodiments, the compatible cells and/or cells lines are non-human animal cells and/or non-human animal lines cells.

In certain embodiments, the compatible cells and/or cell lines are derived from the subject.

In certain embodiments, the compatible cells and/or cell lines are derived from an individual other than the subject.

In certain embodiments, the compatible cells and/or cell lines are identified through the steps of: determining HLA loci associated with the iPSCs and/or iPS cell lines and/or one or more iPSC-derived cells and/or iPSC-derived cell lines from which the composition is manufactured; and matching, by a processor of a computing device, the determined HLA loci with the HLA loci of the subject, wherein a match is an exact match or a partial match.

In certain embodiments, the treatment is administered in one or more doses according to a dosing regimen.

In another aspect, the invention is directed to a composition of matter comprising one or more of (I)-(III) as follows: (I) iPSC-derived Mesenchymal Stem Cells (MSCs), (II) iPSC-derived chondrocytes, and (III) iPSC-derived chondrons, further comprising one or more compatible cell-secreted species, wherein the composition is produced by the method of any one of the preceding claims.

In certain embodiments, the one or more compatible cell-secreted species comprise one or more members selected from the group consisting of collagen, proteoglycans, glycosaminoglycans, exosomes, and microvesicles.

In certain embodiments, the composition is a treatment spray, and/or treatment cream, and/or treatment lotion, and/or a treatment injection.

In certain embodiments, the composition comprises compatible cells, conditioned culture media, and one or more compatible-cell-secreted species, wherein the one or more compatible cell-secreted species are one or more members selected from the group consisting of collagen, proteoglycans, glycosaminoglycans, exosomes, and microvesicles. In certain embodiments, the compatible cells are one or more members selected from the group consisting of iPSCs, MSCs, RPEs, chondrocytes, embryoid bodies, HSCs, blood progenitor cells, and iPSC-derived cells.

In certain embodiments, the composition comprises one or more additives. In certain embodiments, the one or more additives comprises one or more nutrients and/or one or more supplements.

In certain embodiments, the composition comprises an iPS cell and/or cell line retrieved from a biological sample of a subject.

In certain embodiments, the composition comprises compatible cells from a physical repository, wherein the compatible cells are identified as compatible to the subject.

In certain embodiments, the composition comprises compatible cells from a physical repository, wherein the compatible cells are identified as compatible to a particular group of subjects.

In certain embodiments, the compatible cells are identified as compatible with the subject or the particular group of subjects using an identification of cell type indicative of compatibility with the particular subject or particular group of subjects, wherein the identification of cell type indicative of compatibility comprises an HLA match with the particular subject or the particular group of subjects.

In certain embodiments, the composition comprises wherein the composition comprises collagen, proteoglycans, and glycosaminoglycans.

In certain embodiments, the composition comprises chondrons.

In certain embodiments, the composition is formulated internal use (e.g., injection, parenteral, oral, rectal, vaginal etc.).

In certain embodiments, the composition is formulated as an injection.

In certain embodiments, the composition is stored at about −195° C.

In certain embodiments, the composition comprises engineered compatible cells. In certain embodiments, the engineered compatible cells are modified to upregulate and/or downregulate production of one or more cell-secreted species in the composition.

In certain embodiments, the composition comprises compatible cells engineered using CRISPR/Cas9 technology.

In another aspect, the invention is directed to a method of storing a composition tailored for treatment of a subject, said method comprising the steps of: (a) identifying, by a processor of a computing device, as compatible with the subject, one or more compositions derived using compatible cells corresponding to one or both of (i) and (ii) as follows: (i) one or more induced pluripotent stem (iPS) cells and/or iPSC-derived cells, said cells being of one or more HLA types each of which is identified as compatible with the subject, and (ii) one or more iPS cell lines and/or one or more iPSC-derived cell lines, said cell lines being of one or more HLA types each of which is identified as compatible with the subject; (b) labelling, by a processor of a computing device, the one or more compositions with a label, wherein the label comprises information relating to the iPSC and/or iPSC-derived cell and/or IPS cell line and/or IPSC-derived cell line, and/or a classification of the iPSC cell and/or iPSC-derived cell and/or IPS cell line and/or IPSC-derived cell line the composition is derived from; and (c) storing, by a processor of a computing device, a database comprising a data entry corresponding to each label in a physical repository.

In another aspect, the invention is directed to a method of preparing storable iPSC-derived chondrons from iPSC-derived chondrocytes, the method comprising: reacting the iPSC-derived chondrocytes (e.g., mature chondrocytes, more than 30 days in differentiation) in digestion media to produce chondrons; step freezing the chondrons (e.g., by performing a plurality of steps to gradually reduce the temperature in stages prior to introduction to storage in liquid nitrogen); and storing the step-frozen chondrons.

In certain embodiments, the method further comprises thawing the iPSC-derived chondrons to produce viable chondrogenic cells (e.g., via immersion in a hot (e.g., 37° C.) water bath for thawing as rapidly as possible). In certain embodiments, the thawing is performed in the presence of a pericellular matrix (PCM).

In certain embodiments, the method comprises retaining a pericellular matrix prior to the step freezing.

In certain embodiments, the digestion media comprises a collagenase.

Elements of embodiments involving one aspect of the invention (e.g., methods) can be applied in embodiments involving other aspects of the invention (e.g., systems).

### BRIEF DESCRIPTION OF THE DRAWING

The Drawing, which are comprised of at least the following Figures, is for illustration purposes only, not for limitation.

FIG. 1 is a block diagram of an example network environment for use in the methods and systems described herein, according to an illustrative embodiment.

FIG. 2 is a block diagram of an example computing device and an example mobile computing device, for use in illustrative embodiments of the invention.

FIG. 3 is a block diagram showing a method of manufacturing an iPSC-derived chondrogenic composition, according to an illustrative embodiment of the invention.

FIG. 4 is a block diagram showing a method of storing an iPSC-derived chondrogenic composition, according to an illustrative embodiment of the invention.

FIG. 5 is a block diagram showing a method of retrieving one or more produced, labeled and stored iPSC-derived chondrogenic compositions, according to an illustrative embodiment of the invention.

FIG. 6 is a block diagram showing a method of administering an iPSC-derived chondrogenic composition, according to an illustrative embodiment of the invention.

FIG. 7 is a block diagram showing a method of treating a condition in a subject, according to an illustrative embodiment of the invention.

FIG. 8 is a block diagram showing a method of administering an iPSC-derived chondrogenic composition to a subject, according to an illustrative embodiment of the invention.

FIG. 9 shows images of histologically analyzed toluidine stained sections of the right knee of representative rats from the vehicle, positive control, and chondrogenic composition treated rats. (a) (left) The image demonstrates the damage from the surgery in the vehicle treated animal. (b) (middle) The image demonstrates the repair of the cartilage after treatment with chondrocytes (i.e., iPSC-derived chondrocytes prepared by thawing frozen iPSC-derived chondrons) using the chondrogenic compositions prepared according to an illustrative embodiment of the invention. (c) (right) The image demonstrates repair of the cartilage after treatment with FGF18 which stimulates chondrogenesis (positive control).

FIG. 10 demonstrates the results of the gait analysis and change in body weight after administration of chondrogenic compositions to rats with medial meniscal tear (MMT), according to an illustrative embodiment of the invention. (a) (left) The graph demonstrates changes from the gait analysis. The lower the score, the closer to normal. With the chondrocytes (i.e., iPSC-derived chondrocytes prepared by thawing frozen iPSC-derived chondrons), the gait was better compared to the vehicle control. (b) (right) The graph demonstrates changes in weight of rats. The center bar corresponds to the rats that were injected with the chondrogenic compositions, which showed a weight gain compared to the vehicle and the positive control.

FIG. 11 demonstrates the results of the cytokine levels after administration of various experimental compositions to rats with medial meniscal tear (MMT), according to an illustrative embodiment of the invention. (a) IL-6 levels: IL-6 levels have been found to commonly correlate with the severity of the injury. B. IL-1 beta levels: IL-1 beta levels exacerbate damage during chronic disease and acute tissue injury. Thus, a lower the score for either cytokines indicates a lower inflammatory response. Legend: PBS: phosphate buffered saline, HA: hyaluronic acid, MSC: mesenchymal stem cells, MSC/Chond: mesenchymal stem cells/chondrocytes 50:50 mixture, BM-MSC: bone marrow-derived mesenchymal stem cells.

FIG. 12 demonstrates the results of the synovitis score test after administration of various experimental compositions to rats with medial meniscal tear (MMT), according to an illustrative embodiment of the invention. Synovitis is the medical term for inflammation of the synovial membrane. The synovium is the soft tissue that lines the inner surface of the joint and creates the synovial fluid, which lubricates the joint and provides nutrients in the absence of vascularization. The condition of synovitis often occurs from joint damage or disease and causes swelling and is usually very painful when the joint is moved. Synovitis is significantly reduced the very minimal range (0.5) after cell treatments after 28 days (panel (a)) and to an even greater extent to almost normal levels after 56 days (panel (b)). Lower score indicates reduced synovitis. Legend PBS: phosphate buffered saline, HA: hyaluronic acid, MSC: mesenchymal stem cells, MSC/Chond: mesenchymal stem cells/chondrocytes 50:50 mixture, BM-MSC: bone marrow-derived mesenchymal stem cells.

FIG. 13 demonstrates the results of the Medial Tibial Collagen Degeneration score test after administration of various experimental compositions to rats with medial meniscal tear (MMT), according to an illustrative embodiment of the invention. Collagen damage across the medial tibial plateau were quantified and expressed as a percentage of the total tibial surface width. Measurements were made after 28 days and show a reduction after treatments with HA, BN-MSC, MSC, and chondrocytes. Legend: PBS: phosphate buffered saline, HA: hyaluronic acid, MSC: mesenchymal stem cells, MSC/Chond: mesenchymal stem cells/chondrocytes 50:50 mixture, BM-MSC: bone marrow-derived mesenchymal stem cells

FIG. 14 demonstrates the results of the Femoral Cartilage Degeneration score test after administration of various experimental compositions to rats with medial meniscal tear (MMT), according to an illustrative embodiment of the invention. The graphed results represent femoral cartilage degeneration scores. The score includes chondrocyte death/loss, proteoglycan loss, and collagen loss or fibrillation. Zones representing different tissue depth were measured and scored. The individual zones were scored (panels (b)-(g)) and also the sum of the scores of all three zones (panel (a)). The data indicate that all of the treatments had an effect to reduce the degeneration compared to the PBS control. Legend: PBS: phosphate buffered saline, HA: hyaluronic acid, MSC: mesenchymal stem cells, MSC/Chond: mesenchymal stem cells/chondrocytes 50:50 mixture, BM-MSC: bone marrow-derived mesenchymal stem cells.

FIG. 15 is a block diagram showing a method of preparing storable iPSC-derived chondrons from iPSC-derived chondrocytes, according to an illustrative embodiment of the invention.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

“Administration”: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be, within a specific joint (e.g., knee). In some embodiments, administration may be by injection. In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

“Animal”: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

“Biological Sample” or “Sample”: As used herein, the term “sample” or “biological sample”, as used herein, refers to a biological sample obtained or derived from a source of interest, as described herein. In certain embodiments, a source of interest comprises an organism, such as a microbe, a plant, an animal, or a human. In certain embodiments, a biological sample is or comprises biological tissue or fluid. In certain embodiments, a biological sample may be or comprise bone marrow; blood; blood cells; ascites; tissue or fine needle biopsy samples; cell-containing body fluids; free floating nucleic acids (e.g., cell free DNA); sputum; saliva; urine; cerebrospinal fluid, peritoneal fluid; pleural fluid; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc.. In certain embodiments, a biological sample is or comprises cells obtained from an individual. In certain embodiments, obtained cells are or include cells from an individual from whom the sample is obtained. In certain embodiments, a sample is a “primary sample” obtained directly from a source of interest by any appropriate means. For example, in certain embodiments, a primary biological sample is obtained by methods selected from the group consisting of a swab, biopsy (e.g., fine needle aspiration or tissue biopsy), surgery, collection of body fluid (e.g., blood, lymph, feces etc.), etc.. In certain embodiments, as will be clear from context, the term “sample” refers to a preparation that is obtained by processing (e.g., by removing one or more components of and/or by adding one or more agents to) a primary sample. For example, filtering using a semi-permeable membrane. Such a processed “sample” may comprise, for example nucleic acids or proteins extracted from a sample or obtained by subjecting a primary sample to techniques such as amplification or reverse transcription of mRNA, isolation and/or purification of certain components, etc..

“Cancer”: As used herein, the terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

“Carrier”: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

“Chondrocyte therapy composition” or “Chondrogenic composition”: As used herein, the term “chondrocyte therapy composition” or “chondrogenic composition” refers to a composition comprising one or more of (i) to (vi) as follows: (i) iPSC-derived MSCs; (ii) iPSC-derived chondrocytes; (iii) a transcriptome, or portion thereof, of iPSC-derived MSCs; (iv) a transcriptome, or portion thereof, of iPSC-derived chondrocytes; (v) a secretome, or portion thereof, of iPSC-derived MSCs; (vi) a secretome, or portion thereof, of iPSC-derived chondrocytes; and (vii) iPSC-derived chondrons. In certain embodiments, a chondrogenic composition may include chondrocytes and/or chondrons themselves, and/or other cells. In certain embodiments, a chondrogenic composition may include one or more exosomes, and/or one or more microvesicles. A chondrogenic composition may be purified or unpurified. A chondrogenic composition may further comprise one or more substances that are not secreted from a cell (e.g., differentiation media, culture media, additives, nutrients, etc.).

“Chondron”: As used herein, the term “chondron” refers to a chondrocyte and surrounding pericellular matrix (PCM). In certain embodiments, a chondron comprises a plurality of chondrocytes. In some embodiments, a chondron comprises a plurality of chondrocytes clumped together to form a block of cells. In certain embodiments, chondrons may be dissociated into chondrocytes and/or smaller chondrons. In some embodiments, chondrons may be derived directly or indirectly from iPSCs.

“Composition”: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

“Dedifferentiated”: As used herein, the term “dedifferentiated” describes a biological cell that has regressed into an earlier developmental stage (e.g., that of an iPSC or other stem cell) from a differentiated stage (e.g., a cell with more specialized function, e.g., a progenitor cell). For example, a difference between stem cells and progenitor cells is that stem cells can replicate indefinitely, whereas progenitor cells can divide only a limited number of times. The differentiated stage from which the dedifferentiated cell can be produced may be a partially undifferentiated cell. A partially undifferentiated cell is a cell that can differentiate further. An example of a partially undifferentiated cell is a progenitor cell.

“Engineered”: Those of ordinary skill in the art, reading the present disclosure, will appreciate that the term “engineered”, as used herein, refers to an aspect of having been manipulated and altered by the hand of man. In particular, the term “engineered cell” refers to a cell that has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation. In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell.

“Genotype”: As used herein, the term “genotype” refers to the diploid combination of alleles at a given genetic locus, or set of related loci, in a given cell or organism. A homozygous subject carries two copies of the same allele and a heterozygous subject carries two distinct alleles. In the simplest case of a locus with two alleles selected from the DNA nucleotides A, T, G, C, forming pairs, e.g. AA, AT, or TT or AA, AG, GG or AA, AC, CC, etc.

“Genotyping data”: As used herein, the term “genotyping data” refers to data obtained from measurements of a genotype. In certain embodiments, genotyping data describes an individual's phenotype. Genotyping data may be measurements of particular genes (e.g., portions of an individual's genetic sequence, e.g., DNA sequence), SNPs, or variants of SNPs. In certain embodiments, genotyping data is obtained from a multi-gene panel. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

“Human”: In some embodiments, a human is an embryo, a fetus, an infant, a child, a teenager, an adult, or a senior citizen.

“iPSC-derived”: As used herein, the term “iPSC-derived” refers to a composition, or cell, or molecule, or element of a cell which is derived from an induced pluripotent stem cell (iPSC) and/or iPSC cell line. In certain embodiments, the composition, or cell, or molecule, or element of a cell may be derived directly or indirectly from the iPS cell and/or cell line. iPSC-derived cells may include, for example, engineered cells such as iPS cells (and/or cells derived therefrom) that are edited via ZFN (Zinc-finger nucleases), TALEN (Transcription activator-like effector nucleases), CRISPR-Cas9 (Clustered Regularly Interspaced Short Palindromic Repeats, CRISPR-associated protein-9 nuclease), and/or another genome editing technology to result in (or to increase the likelihood of) the upregulation or the downregulation of a cellular component. The upregulated or downregulated cellular component of the genetically modified cells may be, for example, a protein, a nucleic acid, and/or a particular form thereof.

“iPSC-derived composition”: As used herein, the term “iPSC-derived composition” refers to a composition comprising cells, and/or elements of cells, and/or molecules produced by cells, and/or cell-secreted species. The cells may be one or more of iPSCs, iPS cell lines, iPSC-derived cells, and iPSC-derived cell lines.

“Partially un/differentiated”: As used herein, the term “partially un/differentiated” describes a biological cell that, like a state of stem cell, has a tendency to differentiate into a specific type of cell, but is already more specific than a stem cell and is pushed to differentiate into its “target” cell. For example, a difference between stem cells and progenitor cells is that stem cells can replicate indefinitely, whereas progenitor cells can divide only a limited number of times. An example of a partially undifferentiated cell is a progenitor cell.

“Secretome”: As used herein, the term “secretome” refers to one or more substances which are secreted from a cell. In certain embodiments, the secretome refers to the totality of organic and inorganic elements and molecules secreted by a cell, tissue, organ, or organism into its environment (all species secreted at any given moment of time or over a certain period of time). This includes, but is not limited to, secreted proteins, microvesicles, and exosomes. In certain embodiments, secretome refers to the totality of molecules and elements secreted by a cell (all species secreted by the cell at any given moment of time or over a certain period of time). In certain embodiments, secretome refers to a subset of the molecules and elements secreted by a cell (a subset of the species secreted by the cell at any given moment of time or over a certain period of time).

“Subject” or “Individual”: As used herein, the term “subject” or “individual” refers to a human or other animal, or plant. In certain embodiments, subjects are humans and mammals (e.g., mice, rats, pigs, cats, dogs, horses, and primates). In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals are, for example, rodents (e.g., mice, rats, hamsters), rabbits, primates, or swine such as inbred pigs and the like.

“Transcriptome” or “Transcriptome Composition”: As used herein, the term “transcriptome” or “transcriptome composition” refers to a composition comprising one or more transcripts which are generated and/or secreted from a cell, a group of cells, a tissue, an organ, or an organism. In certain embodiments, a transcriptome composition may include one or more types of RNAs (e.g., miRNA, mRNA, tRNA, siRNA, and so forth) , one or more exosomes comprising RNAs, and/or one or more microvesicles comprising RNAs. A transcriptome composition may be purified or unpurified. A transcriptome composition may further comprise one or more substances that are not secreted from a cell (e.g., media, additives, nutrients, etc.).

“Treatment”: As used herein, the term “treatment” (also “treat” or “treating”) refers to any administration of a therapy (e.g., administration of a composition, e.g., via an indicated dosing regimen) that partially or completely alleviates, ameliorates, relieves, inhibits, delays onset of, reduces severity of, and/or reduces incidence of one or more symptoms, features, and/or causes of a particular disease, disorder, defect, and/or condition. In some embodiments, such treatment may be of a subject who does not exhibit signs of the relevant disease, disorder, defect, and/or condition, and/or of a subject who exhibits only early signs of the disease, disorder, defect, and/or condition. Alternatively or additionally, such treatment may be of a subject who exhibits one or more established signs of the relevant disease, disorder, defect, and/or condition. In some embodiments, treatment may be of a subject who has been diagnosed as suffering from the relevant disease, disorder, defect, and/or condition. In some embodiments, treatment may be of a subject known to have one or more susceptibility factors that are statistically correlated with increased risk of development of the relevant disease, disorder, defect, and/or condition.

“Unit dose”: The expression “unit dose” as used herein refers to an amount administered as a single dose and/or in a physically discrete unit of a pharmaceutical composition. In some embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### DETAILED DESCRIPTION

Presented herein are “personalized” iPSC-derived compositions and methods of producing “personalized” iPSC-derived compositions suitable for chondrocyte therapy (e.g., suitable for chondrocyte therapy for cartilage repair), or suitable for other therapies, to be administered to a specific individual and/or specific group of individuals. The iPS cells and/or cell lines, iPSC-derived cells and/or cell lines, and any iPSC-derived chondrogenic compositions derived therefrom, are identified as compatible with a specific individual or specific group of individuals using an identification of a cell type indicative of compatibility such as an HLA match. The compatible iPS cells or cell lines (and/or cells/cell lines derived therefrom) are then retrieved from a managed HLA-indexed (and/or otherwise indexed) repository or are derived from a biological sample of a suitable donor. The retrieved compatible cells are then used to derive the “personalized” iPSC-derived chondrogenic composition, wherein the “personalized” iPSC-derived chondrogenic composition comprises the one or more desired chondrocyte-secreted molecules suitable for chondrocyte therapy of a specific individual and/or specific group of individuals. In certain embodiments, iPSC-derived chondrogenic compositions comprise one or more of (i)-(iii) as follows: (i) Induced Pluripotent Stem Cell (iPSC)-derived Mesenchymal Stem Cells (MSCs), (ii) iPSC-derived chondrocytes, and (iii) iPSC-derived chondrons. Such iPSC-derived chondrogenic compositions, as disclosed herein, may be typed and/or characterized.

In certain embodiments, chondrogenic compositions derived from iPSCs, and/or hematopoietic stem cells (HSCs), and/or blood progenitor cells, and/or mesenchymal stem cells (MSCs), and/or Retinal Pigment Epithelium (RPEs), and/or chondrocytes, and/or embryoid bodies, and/or any other iPSC-derived cells and/or any combinations thereof are useful as therapies to treat various diseases, e.g., rheumatic diseases (e.g., rheumatoid arthritis (RA), osteoarthritis (OA)), cancer (e.g., bone cancer), cartilage damage (e.g., due to injury or congenital defect in elastic cartilage in the ear and nose, due to injury or congenital defect in fibrocartilage found in the vertebral discs, hips, and pelvis, due to injury or congenital defect in hyaline cartilage found between the ribs, in the windpipe, and in joints), arthritis, chondropathy, relapsing polychondritis, osteochondritis dissecans, costochondritis, Chondromalacia patellae, and inflammation. In certain embodiments, collagen, or one or more proteoglycans, or one or more glycosaminoglycans produced by the chondrocytes, is isolated and used in the treatment of disease or other therapy.

In certain embodiments, chondrocytes and/or chondrons derived from iPSCs, and/or hematopoietic stem cells (HSCs), and/or blood progenitor cells, and/or mesenchymal stem cells (MSCs), and/or Retinal Pigment Epithelium (RPEs), and/or embryoid bodies, and/or any other iPSC-derived cells and/or any combinations thereof are grown and/or cultured in a mold (e.g., ex vivo) that is sized and shaped to produce desired macroscopic cartilage structures (e.g., portion of or full meniscus, portion of or full septal nasal cartilage, portion of or full elastic cartilage of the ear and nose, portion of or full fibrocartilage found in the vertebral discs, hips, and pelvis, portion of or full hyaline cartilage found between the ribs, in the windpipe, and in joints). As used herein, “macroscopic” means on a scale of at least about 20 um, up to much larger sizes, e.g., on the scale of centimeters, decimeters, or larger. In certain embodiments, the macroscopic cartilage structures may be produced from the retrieved compatible cells (e.g., wherein the retrieved compatible cells comprise chondrocytes). In certain embodiments, the macroscopic cartilage structures may be produced from chondrocytes derived from the retrieved compatible cells (e.g., hematopoietic stem cells (HSCs), and/or blood progenitor cells, and/or mesenchymal stem cells (MSCs), and/or Retinal Pigment Epithelium (RPEs), and/or embryoid bodies, and/or any other iPSC-derived cells and/or any combinations thereof). Following growth of the macroscopic structure (e.g., ex vivo), the structure may be applied as treatment (e.g., via implantation) of damaged, diseased, or abnormal cartilage tissue of an individual. The macroscopic structure may replace or supplement existing cartilage tissue in the individual. The mold may be made, for example, from a polymeric material or other nontoxic and/or biocompatible material.

In certain embodiments, macroscopic cartilage structures are produced from chondrocytes via 3D printing. For example, the iPSC-derived composition (e.g., comprising the retrieved compatible cells (e.g., chondrocytes, and/or MSCs, and/or chondrons)) may be 3D printed into desired three-dimensional macroscopic cartilage structures (e.g., portion of or full meniscus, portion of or full septal nasal cartilage, portion of or full elastic cartilage of the ear and nose, portion of or full fibrocartilage found in the vertebral discs, hips, and pelvis, portion of or full hyaline cartilage found between the ribs, in the windpipe, and in joints), In certain embodiments, the iPSC-derived chondrogenic composition used to 3D-print macroscopic cartilage structures may be produced from the retrieved compatible cells (e.g., wherein the retrieved compatible cells comprise chondrocytes). In certain embodiments, the iPSC-derived chondrogenic composition used to 3D-print macroscopic cartilage structures may be produced from chondrocytes derived from the retrieved compatible cells (e.g., hematopoietic stem cells (HSCs), and/or blood progenitor cells, and/or mesenchymal stem cells (MSCs), and/or Retinal Pigment Epithelium (RPEs), and/or embryoid bodies, and/or any other iPSC-derived cells and/or any combinations thereof). Following 3D-printing of the macroscopic structure (e.g., ex vivo), the structure may be applied as treatment (e.g., via implantation) of damaged, diseased, or abnormal cartilage tissue of an individual. The macroscopic structure may replace or supplement existing cartilage tissue in the individual. Various techniques for creating organic structures via 3D printing have been documented. See, for example, Bose S., et al., Bone tissue engineering using 3D printing, Materials Today, 16 (12), 2013; Lee M., and Wu B. M., Recent Advances in 3D Printing of Tissue Engineering Scaffolds, in: Liebschner M. (eds) Computer-Aided Tissue Engineering, Methods in Molecular Biology (Methods and Protocols), 868, 2012, Humana Press, Totowa, N.J.; Ventola M. C., Medical Applications for 3D Printing: Current and Projected Uses, Pharmacy and Therapeutics, 39(10), 2014. For example, a previous technique may involve the layer-by-layer construction of a three-dimensional cell scaffold which is seeded with cells of interest. Such steps, and/or adaptions of such steps may be conducted for the techniques described herein.

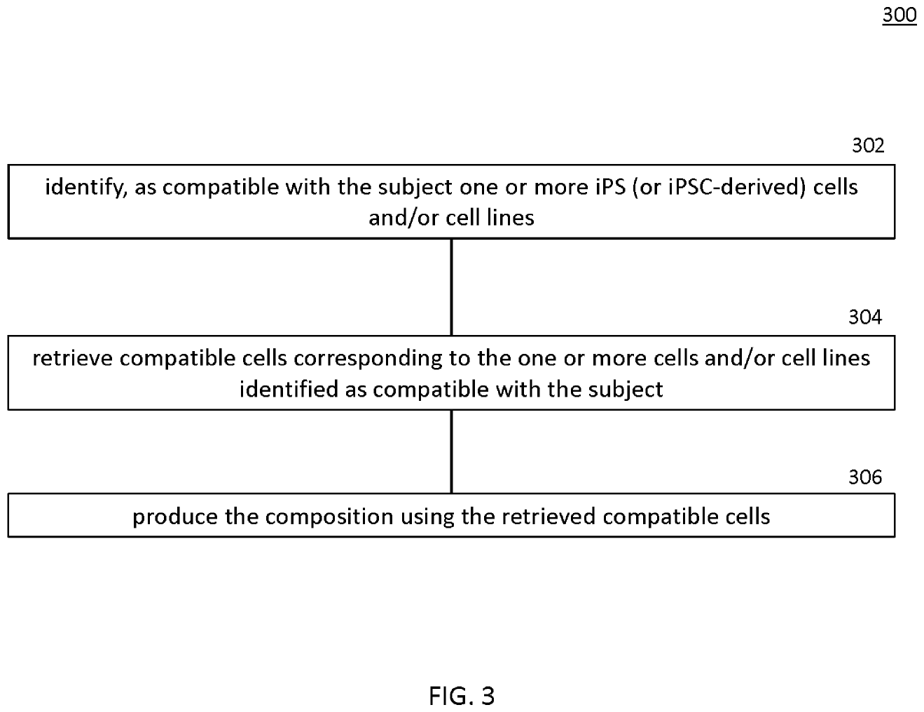

FIG. 3 is a block diagram showing a method 300 of manufacturing an iPSC-derived chondrogenic composition, according to an illustrative embodiment of the invention. In one step 302 the induced pluripotent stem (iPS) cells and/or iPSC-derived cells are identified as compatible with the particular subject or particular group of subjects. In certain embodiments, the iPS and/or iPSC-derived cells may belong to one or more cell types (e.g., HLA types), each of which is compatible with the particular subject or group of subjects. In certain embodiments one or more iPS cell lines and/or one or more iPSC-derived cell lines may also be identified, said cell lines being of one or more types (e.g., HLA types) each of which is compatible with the particular subject or group of subjects. In certain embodiments, the compatible cells and/or cell lines may be derived from the subject (e.g., autologous). In certain embodiments, the compatible cells and/or cell lines may be from an individual other than the subject (e.g., allogeneic). In another step 304, the compatible cells corresponding to the one or more cells and/or cell lines identified as compatible with the particular subject or particular group of subjects are retrieved. The iPSC-derived chondrogenic composition is then produced 306 using the retrieved compatible cells. In certain embodiments, the iPSC-derived chondrogenic composition comprises chondrocytes and one or more desired compatible-cell-secreted species (e.g., molecules and/or biological elements), (e.g., collagen, proteoglycans etc.) suitable for cartilage repair of the subject.

The techniques described herein allow for the tuning of chondrogenic compositions to a specific individual or a specific group of individuals, thus enabling improved methods of chondrocyte therapy, e.g. due to an enhanced compatibility of the specific individual or group of individuals with the cells from which the desired chondrogenic composition is derived. Also, allogeneic iPS cells and/or cell lines that are compatible with a large portion of a specific population, e.g. super donors, can be prepared and stored in advance for large groups of individuals. These super donor-derived chondrogenic compositions can then be made immediately available to people who need them, thus reducing production times of the iPSC-derived chondrogenic compositions.

iPSCs, or cells differentiated from iPSCs, can be made to produce a desired chondrogenic composition, e.g., which comprises desired proteoglycans or glycosaminoglycans. For example, chondrogenic composition can be produced from iPSCs of a super donor cell line. Chondrogenic compositions can also be produced from MSCs, chondrocytes, or other cell types derived from iPSCs. In certain embodiments, allogeneic iPSCs (and/or cells derived therefrom) and/or allogeneic iPSC-derived chondrogenic compositions can be prepared and stored for large groups of individuals. Allogeneic iPSCs (and/or cells derived therefrom) and/or iPSC-derived chondrogenic compositions can be made in advance so that they are ready when people need them. For example, the iPSCs, and/or iPSC-derived cells and/or iPSC-derived chondrogenic compositions can be frozen or lyophilized and stored for later use.

In certain embodiments, iPSCs (and/or cells derived therefrom) and/or iPSC-derived chondrogenic compositions can be lyophilized to manufacture a more concentrated solution or composition. In certain embodiments, iPSCs, or cells differentiated from iPSCs, can be engineered using various technologies (e.g., CRISPR/Cas9) to upregulate production of one or more desired proteoglycans or glycosaminoglycans in the chondrogenic composition. For example, in certain embodiments, an iPS cell (and/or cells derived therefrom) may be edited via CRISPR (e.g., CRISPR-Cas9 genome editing and/or gene transfer) to remove, replace, and/or edit one or more genes to result in (or to increase the likelihood of) the upregulation of one or more desired proteoglycans or glycosaminoglycans in the iPSC-derived chondrogenic composition.

In certain embodiments, provided herein is a managed repository of chondrogenic compositions, hematopoietic stem cell (HSC) lines and/or blood progenitor cell lines, RPE lines, MSC lines, chondrocyte lines and/or other cell lines derived from induced pluripotent stem cells (iPSCs) (e.g., embryoid bodies or other tissues formed from iPSCs). In certain embodiments, the chondrogenic compositions, HSC lines, blood progenitor cell lines, embryoid bodies, RPE lines, MSC lines, chondrocyte lines, iPSC lines and/or iPSC-derived cell lines has corresponding data comprising a set of characterized HLA loci, said corresponding data being stored in a searchable database for retrieval of one or more matching physical cell lines and/or chondrogenic compositions upon query. The repository may comprise a bank of cells (e.g., iPSCs, HSCs, blood progenitor cells, embryoid bodies, RPEs, MSCs, chondrocytes, other iPSC-derived cells), and/or compositions produced from cells, for each of a set of HLA types. This allows identification and provision of existing compatible iPSC-derived chondrogenic compositions, iPSCs, embryoid bodies, RPEs, MSCs, chondrocytes, HSCs, blood progenitor cells, and/or other iPSC-derived cells for a particular subject or group of subjects. The iPSC-derived chondrogenic composition—and allogeneic cell lines (e.g., iPSC lines, MSC lines, RPE lines, chondrocyte lines, HSC lines, blood progenitor cell lines, other iPSC-derived cell lines) suitable for deriving chondrogenic compositions—can be used to formulate compositions for administration topically or internally (e.g., injection, parenteral, oral, rectal, vaginal etc.) to regenerate, and/or treat cartilage in patients with damaged, diseased, or otherwise abnormal cartilage. For example, iPSCs, iPSC-derived cells (e.g., HSCs, blood progenitor cells, embryoid bodies, RPEs, MSCs, chondrocytes, other iPSC-derived cells), iPSC-derived composition (e.g., chondrogenic composition), and/or combinations therefrom can be administered via an injection (e.g., subcutaneous, intramuscular, etc.) to anatomical areas that have low vasculature (e.g., around joints) to aid in repair of the damaged cartilage in the region. In certain embodiments, the administered solution of cells, compositions and/or combinations therefrom may include additives (e.g., nutrients to keep cells alive/active before, during, and/or after administration, carriers, fillers etc.).

### Human Leukocyte Antigen

The characterized iPS cells and/or cell lines and/or compositions derived therefrom are stored in the repository that is indexed using the Human Leukocyte Antigen (HLA). In certain embodiments, the iPS cells and/or cell lines and/or compositions derived therefrom are characterized and indexed as super donor cell lines via HLA mapping (e.g., HLA typing and/or matching). In certain embodiments, multiple HLA loci may be characterized and indexed for each of the various iPS cells and/or cell lines and/or cells derived therefrom and/or compositions derived therefrom.

The HLAs in humans are major histocompatibility complex (MHC) proteins that function to regulate the immune system. HLA genes are highly polymorphic and may be broadly divided into Class I and Class II. For example, Class I in humans may be found on all nucleated cells and platelets. On the other hand, HLA Class II (constitutive expression), for example, may be restricted to specialized cells of the immune system (e.g., macrophages, B cells, etc.).

HLA Class I, for example, may include HLA-A, B, and C genes. In certain embodiments, HLA Class I may be co-dominantly expressed on the cell surface and may present peptides derived from internal cellular proteins to the T cell receptor of CD8 T cells. For example, these proteins may be involved in the immune response against intracellular parasites, viruses, and cancer.

In certain embodiments, HLA Class I may have a heterodimeric protein structure, with a polymorphic alpha chain and a common beta-2 microglobulin. In certain embodiments, the alpha chain may be composed of 3 extracellular domains: α1, α2, and α3.

HLA Class II, for example, may include DR, DQ, and DP genes. In certain embodiments, HLA Class II may be co-dominantly expressed. In certain embodiments, HLA Class II may have a heterodimeric protein structure, with a polymorphic beta chain and a much less polymorphic alpha chain. In certain embodiments, both chains may be composed of two (2) extracellular domains (α1, α2, and β1, β2). For example, the α1 and β1 domains may create a peptide binding groove which presents processed peptides, from extracellular protein, to CD4+ T cells. In certain embodiments, HLA Class II may be involved in the immune response against extracellular infectious agents and non-self HLA molecules.

In certain embodiments, each HLA allele may be identified by letters indicating “locus” (e.g., A, B, C, DR, DQ, and DP) and individual specificity may be defined by a number following the locus (e.g., A1, B27, DR8, etc.). Specificities can be defined using antisera (antibodies). In certain embodiments, HLA specificities may also be determined using genetic analysis by identifying the presence/absence of the gene encoding the HLA protein. For example, Class II molecular specificities may be identified at the level of the gene encoding a particular chain (α or β).

### HLA Typing

The stem cells and/or stem cell lines (e.g., iPSCs) and/or cells derived therefrom and/or compositions derived therefrom stored in the physical repository may be characterized and indexed using various characteristics of the samples (e.g., cells). In certain embodiments, the stem cells and/or cell lines and/or cells derived therefrom and/or compositions derived therefrom may be characterized and indexed using HLA type.

As discussed in Taylor et al., Cell Stem Cell 11, Aug. 3, 2012, pp. 147-152, the contents of which are incorporated herein by reference, HLA-mismatched iPSCs can cause immunological rejection and therefore limit therapeutic potential. iPSCs derived directly from patients (autologous iPSCs) can result in matched HLA type and reduce risk of transplant rejection. However, generation of autologous iPSCs for individual patients is costly and time-consuming. Alternatively, allogeneic iPSC cell lines with HLA types that do not trigger strong reactions can be prepared and used for large groups of individuals.

HLA typing or HLA matching is used to determine the HLA type of an individual. The HLA type of an individual comprises a pair of co-expressed haplotypes, each corresponding to a set of HLA genes (e.g., an HLA-A, an HLA-B, and an HLA-DR gene). In certain embodiments, genetic recombination and environmental factors result in linkage disequilibrium with respect to inheritance of HLA gene combinations. For example, certain combinations of HLA alleles (e.g., combinations of HLA-A, -B, and -DR genes) are favored, whereas other combinations do not exist.

HLA typing may be performed at a protein level but may also be performed at the DNA level, for example by amplifying the DNA via polymerase chain reaction (PCR), or other DNA identification and amplification technologies. For example, HLA typing may be performed using sequence specific oligonucleotides (SSO). In certain embodiments, SSO-based HLA typing may use generic primers to amplify large amounts of HLA alleles, for example, HLA-A, via PCR or other DNA amplification technologies. The dsDNA is separated into single strands and allowed to interact with the single strand specific oligonucleotide probes. In certain embodiments, such probes may be bound to a solid matrix. For example, the pattern of the bound probes may be used to determine the HLA type of the specimen. In certain embodiments, HLA typing may be performed using sequence specific primers (SSP). For example, in SSP-based HLA typing amplifies DNA that matches the primers. Antibodies may also been used for HLA typing, but may have the disadvantage of cross-reacting with multiple HLA epitopes (e.g. HLA-A2, A9 and A28).

Because the iPSC lines, MSC lines, RPE lines, chondrocyte lines, HSC lines, blood progenitor cell lines, and/or other iPSC-derived cell lines are characterized by HLA type, an iPSC line, MSC line, RPE line, chondrocyte line, HSC line, blood progenitor cell line, and/or other iPSC-derived cell line can be identified as suitable for a given patient with a compatible HLA type, with low, reduced, or zero chance of a compatible cell-derived composition rejection. In certain embodiments, the bank of iPSCs, embryoid bodies, MSCs, RPEs, chondrocytes, HSCs, blood progenitor cells, and/or other iPSC-derived cells is comprehensive in that it contains a variety of HLA types covering a significant proportion (e.g., at least 85%, at least 90%, or at least 95%) of a given population. In certain embodiments, the iPSC lines, MSC lines, RPE lines, chondrocyte lines, the HSC lines, blood progenitor cell lines, other iPSC-derived cell lines and/or chondrogenic compositions in the bank and/or the iPS cell lines and/or embryoid bodies from which the MSCs, RPEs, chondrocytes, HSCs, blood progenitor cells, other iPSC-derived cells, and/or the chondrogenic compositions are derived, are characterized as super donor cell lines (e.g., via HLA mapping). Thus, suitable cells (e.g., iPSCs, iPSC-derived cells), cell lines (e.g., iPSC lines, iPSC-derived lines), and/or iPSC-derived chondrogenic compositions for treatment may be quickly identified and made available to patients over a wide swath of a given population upon demand, without the difficult, time consuming process of identifying a matching donor.

### Super Donor:

The term “super donor” is a term used to describe HLA types that do not trigger strong rejection reactions. Such allogeneic (derived from donors other than the patient) iPSC lines can be made in advance and can be ready for use when needed. Using super donors, fewer allogeneic lines will likely be needed to serve a population. iPSCs can be obtained from healthy volunteer donors of blood group O that are selected to maximize the opportunity for HLA matching. Clinical grade iPSC lines can be expanded and differentiated for use in a large number of subjects. Nakajima et al., Stem Cells 25, 2007, pp. 983-985, the contents of which are incorporated by reference herein, discusses HLA matching estimations in a hypothetical bank of human embryonic stem cell lines in the Japanese population, and calculated that a large proportion of patients were able to find at least one HLA matched donor at three loci of HLA-A, HLA-B, and HLA-DR for transplantation therapy.

### Applications of HLA Typing

The HLA type of a sample (e.g., cells, organs, and/or tissue) may be used in determining compatibility between organ donors and recipients. Samples which match the HLA type of a recipient (e.g., patient) are more likely to not illicit an immune response (e.g., rejection) after the sample is transplanted to the recipient. In certain embodiments, matching is performed on the basis of 3 or more loci on the HLA gene to prevent a strong immune response in the recipient post transplantation. In certain embodiments, at least 3 HLA loci are required to match between the donor and the recipient to prevent a strong immune response in the recipient post transplantation. In certain embodiments, at least 3, or at least 4, or at least 5, at least 6, or at least 7, or at least 8, or at least 9 major sites (e.g., loci) are required to match between the donor and the recipient to prevent a strong immune response in the recipient post transplantation.

Many registry donors have been tested by serological (e.g., HLA mapping using antigens) methods, though often without documentation regarding which antigens were tested. While the majority of hematopoietic progenitor cell transplant candidates have been tested by molecular (DNA-based) methodologies, the nomenclature of antigens (serology) and alleles (DNA) is in some cases not concordant. Thus, the characterized and indexed (e.g., HLA indexed (e.g., using standard nomenclature)) iPS cells and/or cell lines and/or cells derived therefrom and/or compositions derived therefrom, described herein, may be used to efficiently and accurately searched using the corresponding database to quickly find matching HLA samples for implantation. For example, the HLA indexed and matched iPS cells and/or cell lines and/or cells derived therefrom and/or compositions derived therefrom may be used in treatment of various diseases. In certain embodiments, these cells and/or cell lines may be used in the treatment cancer (e.g., leukemia, lymphoma, bone cancer, and the like). In certain embodiments, these cells and/or cell lines may be used in stem cell transplantation.

The HLA-indexed repository may also be used for various purposes. For example, other clinical applications of HLA typing may include disease risk assessment, pharmacogenomics, immunotherapy, infectious disease vaccines, and tumor vaccines. In certain embodiments, the cells and/or cell lines stored and indexed in the repository may be used in cosmetic surgery, for example cartilage grafts. Long-term transplant and graft survival is correlated to the degree of HLA antigen mismatch for both solid organ and bone marrow transplant.

HLA matched cells and/or cell lines may also be used in the treatment of various diseases. Certain diseases may have a strong association with certain specific HLA types. For example, HLA associations with diseases include ankylosing spondylitis and acute anterior uveitis (HLA-B27); birdshot retinopathy (HLA-A29); Behget's Disease (HLA-B51); psoriasis (HLA-Cw6); celiac disease (HLA-DQ2,8); narcolepsy (HLA-DR15, DQ6); diabetes (HLA-DR3,4-DQ2,8); and rheumatoid arthritis (HLA-DR4). In certain embodiments, the data entries in the HLA database corresponding to specific samples (e.g., cells and/or cell lines in the physical repository) may incorporate information regarding their specific HLA types to recognize their strong associations with certain diseases.

HLA type may also be associated with allergy or hypersensitivity to a medication. For example, severe allergic or hypersensitivity reaction to drugs in Stevens-Johnson Syndrome (SJS) and toxic epidermal necrolysis (TEN) may be associated with HLA type. The physical repository of cells and/or cells lines and corresponding database may be used to identify allergies and sensitivities in the patients (e.g., sometimes unknown to the patient). In certain embodiments, HLA typing allows risk stratification of the patients. In certain embodiments, drugs that are associated with hypersensitivity reactions (e.g., antiepileptic agents, allopurinol, nevirapine, anti-inflammatories in oxicam family, and sulfonamides) may be studied using the cells and/or cell lines and/or cells derived therefrom stored in the repository. Further, these studies can be performed in vitro and/or ex vivo prior to implantation.

HLA typing may be used for vaccine development. The HLA-indexed cells and/or cell lines and/or cells derived therefrom and/or compositions derived therefrom described herein may be used to develop such vaccines. In certain embodiments, vaccines producing cellular immunity require peptide HLA binding. For example, vaccine trials use peptides binding to common HLA alleles. After proof-of-principal, trials may include peptides binding to other HLA alleles. In certain embodiments, cells with the common HLA allele, and cells with other HLA alleles may be selected from the back of stem cells and/or cell lines stored in the repository.

HLA typing can also be informative for compatibility of individuals. For example, studies have found that husbands and wives have fewer HLA matches than expected. The HLA genes (HLA-A, HLA-B, and HLA-DRB1) regulate the immune system, and thus determine the microbes that the immune system attacks. As a non-limiting example, the HLA genes therefore regulate a subject's smell by governing the non-human microbes associated with that subject and therefore can affect the attraction between subjects based on smell, among other things. Given the association between HLA type and long-term compatibility, it may be possible to predict the likelihood of companionship between two individuals. In some embodiments, the present disclosure teaches a method of querying and retrieving data entries of a database matching queried HLA loci for compatibility or companionship for a given subject with other individuals.

### HLA-Indexed Bank

The bank of iPS cells and iPSC-derived compositions (e.g., IPS cells and/or cell lines and/or cells derived from iPSCs (e.g., HSCs and/or blood progenitors) and/or chondrogenic compositions derived from iPSCs) is a comprehensive indexed repository in that it contains a variety of HLA types covering a significant proportion (e.g., at least 85%, at least 90%, or at least 95%) of a given population, indexed by HLA type. In certain embodiments, the HSC lines and/or blood progenitors in the bank (and/or the iPS cell lines and/or embryoid bodies from which the HSCs and/or blood progenitors are derived), may be characterized as super donor cell lines (e.g., via HLA mapping). Thus, it is possible to obviate the need for bone marrow registries and/or other donor registries, since suitable cells for transplantation may be quickly identified and made available to patients over a wide swath of a given population upon demand, without the difficult, time consuming process of identifying a matching blood marrow donor.

The bank may provide access to reserves of immortalized iPSCs from which chondrogenic compositions can be derived—iPSCs and chondrogenic compositions derived from iPSCs may be prepared in advance for commonly-used/matched HLA types (e.g., HLA super donors matching higher percentages of the population) so that cells and/or compositions are available immediately upon need. HSCs may also be produced for a particular patient upon identification of a matching iPSC line. Furthermore, in certain embodiments, reserves of embryoid bodies, corresponding to characterized iPSC lines, are stored in the bank. In certain embodiments, HLA super donor lines are physically represented in the bank by embryoid bodies (characterized as HLA super donor lines). These embryoid bodies may be used to make iPSCs or iPSC-derived cells. The provided bio-repository or bank may be queried using one or more queries to retrieve data entries corresponding to one or more cells and/or compositions in the repositories as described , for example in International Application No. PCT/US17/67272 entitled “HLA-Indexed Repository Of iPSCs And iPSC-Derived Stem Cells, And Related Systems And Methods” filed on Mar. 14, 2018, the contents of which are hereby incorporated by reference in their entirety.

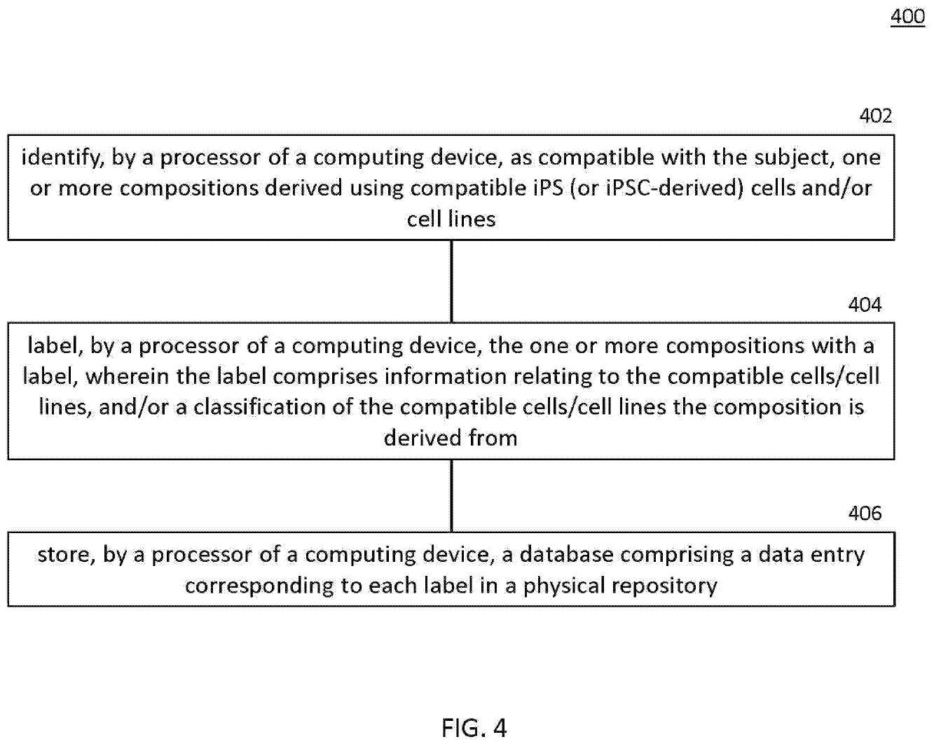

FIG. 4 is a block diagram showing a method 400 of storing an iPSC-derived chondrogenic composition, according to an illustrative embodiment of the invention. In one step 402, one or more iPSC-derived chondrogenic compositions derived using compatible cells are identified, by a processor of a computing device, as compatible with the particular subject or particular group of subjects. In certain embodiments, the compatible cells correspond to one or more iPS (or iPSC-derived (e.g., MSC, HSC, RPE and the like)) cells and/or cell lines, said cells and/or cell lines being of one or more types (e.g., HLA type) each of which is identified as compatible with the particular subject or group of subjects. In another step, 404, the one or more iPSC-derived chondrogenic compositions are labeled, by a processor of a computing device, with a label. In certain embodiments, the label may be a digital label, wherein the label comprises information relating to the iPS and/or iPSC-derived cell and/or cell line, and/or a classification of the iPS cell and/or cell line (e.g., HLA loci) the iPSC-derived chondrogenic composition is derived from. The one or more labeled iPSC-derived chondrogenic compositions are then stored (406), by a processor of a computing device, in a database comprising multiple data entries. Each data entry in the databased corresponds to each labeled iPSC-derived chondrogenic compositions (e.g., or other labeled entities like cells, cell lines, other compositions and the like) stored in a physical repository.

In certain embodiments, the bank may provide access to reserves of immortalized iPSCs from which MSCs, chondrocytes, and/or chondrogenic compositions can be derived. MSCs, chondrocytes and/or cartilage tissue expressing specific proteoglycans and glycosaminoglycans may be prepared in advance for commonly-used/matched HLA types (e.g., HLA super donors matching higher percentages of the population) so that the compositions and tissue are available for treatment and/or implantation immediately upon need. These compositions may also be produced for a particular patient upon identification of a matching iPSC line and/or iPSC-derived cell line.

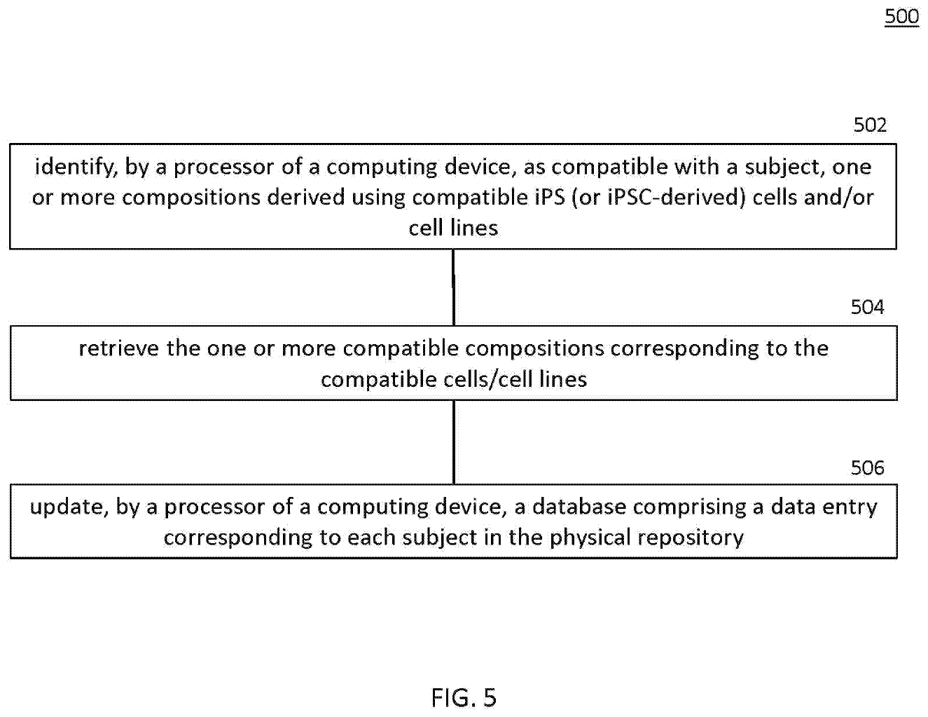

FIG. 5 is a block diagram showing a method 500 of retrieving one or more produced, labeled and stored iPSC-derived chondrogenic compositions, according to an illustrative embodiment of the invention. In one step 502, one or more iPSC-derived chondrogenic compositions are identified, by a processor of a computing device, as compatible with a particular subject or particular group of subjects. The one or more iPSC-derived chondrogenic compositions are derived using one or both of (i) and (ii) as follows: (i) one or iPS cells and/or iPSC-derived cells, said cells being of one or more types (e.g. HLA type) each of which is identified as compatible with the particular subject or group of subjects, and (ii) one or more iPS cell lines and/or one or more iPSC-derived cell lines, said cell lines being of one or more types each of which is identified as compatible with the particular subject or group of subjects. In a following step 504, the one or more compatible iPSC-derived chondrogenic compositions corresponding to the one or more iPS and/or iPSC-derived cells and/or cell lines identified as compatible with the particular subject or particular group of subjects are retrieved (e.g., from the physical repository in which the one or more iPSC-derived chondrogenic compositions are stored). The database data entry of each subject of the group of subjects is then updated (506), by a processor of a computing device. The update to the data entry corresponding to each subject may include identification information (e.g., label information) regarding the one or more iPSC-derived chondrogenic compositions in the physical repository that each subject is compatible with.

Furthermore, in certain embodiments, reserves of embryoid bodies, corresponding to characterized iPSC lines, are stored in the bank. In certain embodiments, HLA super donor lines are physically represented in the bank by embryoid bodies (characterized as HLA super donor lines). These embryoid bodies may be used to make MSCs, and/or chondrocytes that are used to express the collagen and desired proteoglycans and glycosaminoglycans, used to formulate the chondrogenic composition.

The characterized iPSCs and/or embryoid bodies comprising embryonic stem cells (e.g., undifferentiated pluripotent cells) can be differentiated into MSCs and/or chondrocytes and made to produce various chondrogenic compositions in the presence of appropriate culture and/or differentiation media. In certain embodiments, the characterized cell types contained in the physical bank include any one or more of the following: iPSCs, embryoid bodies, HSCs, blood progenitor cells, mature hematopoietic cells, MSCs, RPEs, chondrocytes, and/or other iPSC-derived cells (e.g., that may be differentiated into chondrocytes that produce chondrogenic compositions).

Matching HLA type may involve, for example, querying and retrieving data entries of a database matching queried HLA loci. In certain embodiments, this comprises receiving, by a processor of a computing device (e.g., a server), a data entry for an individual for which a matching iPSC line, and/or MSC line, and/or chondrocyte line, and/or RPE line, and/or HSC line, and/or blood progenitor line, and/or any other iPSC-derived cell line, and/or iPSC-derived chondrogenic composition is desired, the data entry comprising a set of characterized HLA loci corresponding to the individual [e.g., identification (e.g., by processing and analyzing (e.g. by serology, by PCR) samples from the individual (e.g., blood samples)) of each of a set of at least 3 given loci (e.g., HLA-A, HLA-B, and HLA-DRB (e.g., HLA-DRB1)), e.g., at least 9 given loci (e.g., HLA-A, HLA-B, HLA-C, HLA-DRB1, HLA-DRB3, HLA-DRB4, HLA-DRB5, HLA-DQB1, HLA-DPB1), e.g., at least 3, 4, 5, 6, 7, 8, or 9 members selected from this group of nine loci]; and retrieving, by the processor, one or more data entries of a database representative of cells (e.g., iPS cells in the physical repository and/or embryoid bodies, MSCs, RPEs, chondrocytes, HSCs, blood progenitor cells, and/or other cells from a cell line derived from iPSCs), and/or iPSC-derived chondrogenic compositions matching (e.g., exactly matching, partially matching, identified as compatible with (e.g., compatible HLA types), etc.) the queried HLA loci (e.g., determining the corresponding bar code or other identifier for the iPSCs, and/or iPSC-derived cells, and/or embryoid bodies corresponding to the data entry, thereby allowing retrieval of desired stem cells and/or chondrogenic compositions from the repository and/or retrieval of identifying information corresponding to a desired iPSC cell line matching the queried HLA loci). iPSC-derived chondrogenic compositions may be produced from immortalized iPSC lines (e.g., that are differentiated into chondrocytes) at will and made available for ready access when needed—no additional harvesting of samples are required to produce additional iPSC-derived chondrogenic compositions.

The repository/bank of cells and compositions may comprise a storage system comprising an insulated container equipped with environmental control system (for control of temperature, humidity, pressure, and the like) suitable to store cells (e.g., iPSCs, embryoid bodies, RPEs, chondrocytes, MSCs, HSCs, blood progenitor cells, mature hematopoietic cells, and/or other iPSC-derived cells), and chondrogenic compositions (e.g., derived directly or indirectly from iPSC-derived MSCs, and/or chondrocytes) for a period of time. The repository/bank may also include one or more processors (e.g., of a server) and/or related software to manage inventory, as well as a sample location system and/or retrieval system for identification/retrieval of cells and/or specific chondrogenic compositions from a matched cell line. iPSCs may be produced from blood samples (or other biological substance sample, e.g., saliva, serum, tissue, cheek cells, cells collected via a buccal swab, urine, and/or hair), then labeled (physically and/or digitally), logged in an inventory database, and stored in the repository for ongoing and/or future use. MSCs, RPEs, chondrocytes, HSCs, blood progenitor cells, mature hematopoietic cells and/or other cell types may be produced from iPSCs via known methods. Further, the MSCs, RPEs, chondrocytes, HSCs, blood progenitor cells, mature hematopoietic cells and/or other cell types may be converted to each other via known methods (e.g., an iPSC may be differentiated into a chondrocyte, or a blood progenitor cell may be dedifferentiated into a stem cell (e.g., an iPS cell) which may then be differentiated into a chondrocyte). These iPSCs or iPSC-derived cells are differentiated into chondrocytes that are made to produce desired secretomes and formulated into compositions. The iPSC-derived cells and/or chondrogenic compositions may also be labeled (physically and/or digitally), logged in the inventory database, and stored in the repository for ongoing and/or future use.

The repository/bank of cells may be used in systems and methods for regeneration, treatment, and/or enhancement of subjects in need of cartilage therapy. For example, the repository/bank of cells comprise iPSCs and/or embryoid bodies corresponding to/produced from iPSC lines, wherein MSCs, RPEs, chondrocytes, HSCs, blood progenitor cells, and/or other cell types are derived from/produced from the iPSCs and/or embryoid bodies, and the MSCs and/or chondrocytes derived therefrom are utilized to derive specific secretomes that are formulated into chondrogenic compositions, and the chondrogenic compositions are administered to subjects at risk of or having a disease, traumatic injury, and/or condition, such as any of the following: rheumatic diseases (e.g., rheumatoid arthritis (RA), osteoarthritis (OA)), cancer (e.g., bone cancer), cartilage damage (e.g., repair damage due to injury or congenital defect in elastic cartilage in the ear and nose, repair damage due to injury or congenital defect in fibrocartilage found in the vertebral discs, hips, and pelvis, repair damage due to injury or congenital defect in hyaline cartilage found between the ribs, in the windpipe, and in joints), arthritis, chondropathy, relapsing polychondritis, osteochondritis dissecans, costochondritis, Chondromalacia patellae, and inflammation.

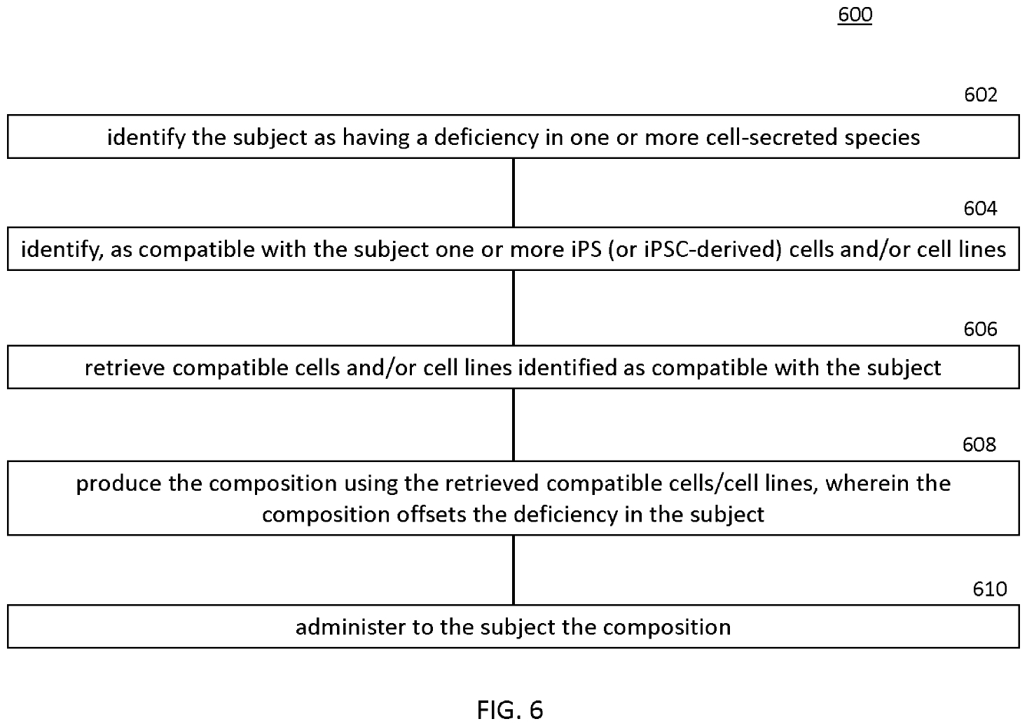

For example, FIG. 6 is a block diagram showing a method 600 of administering an iPSC-derived chondrogenic composition tailored for treatment of a particular subject or particular group of subjects, according to an illustrative embodiment of the invention. In one step 602, the particular subject or particular group of subjects as having a deficiency in one or more cell-secreted species (e.g., one or more chondrocyte-secreted molecules and/or chondrocyte-secreted biological elements) is/are identified. In a second step 604, one or both of (i) and (ii) as follows: (i) one or more induced pluripotent stem (iPS) cells and/or iPSC-derived cells, said cells being of one or more types each of which is compatible with the particular subject or group of subjects, and (ii) one or more iPS cell lines and/or one or more iPSC-derived cell lines, said cell lines being of one or more types each of which is compatible with the particular subject or group of subjects are identified as compatible with the particular subject or particular group of subjects. Following identification, in step 606, the compatible cells corresponding to the iPS and/or iPSC-derived cells and/or cell lines identified as compatible with the particular subject or particular group of subjects are retrieved (e.g., from a physical repository). The iPSC-derived chondrogenic composition is then produced (608) using the retrieved compatible cells. The iPSC-derived chondrogenic composition produced is engineered and/or selected such that it offsets the deficiency in the particular subject or particular group of subjects (e.g., wherein the iPSC-derived chondrogenic composition comprises the identified one or more deficient cell-secreted species [e.g., chondrocyte-secreted molecules and/or chondrocyte-secreted biological elements, (e.g., the one or more proteoglycans and/or glycosaminoglycans) identified as deficient in the subject]. The iPSC-derived chondrogenic composition is then administered (610) to the subject or group of subjects.

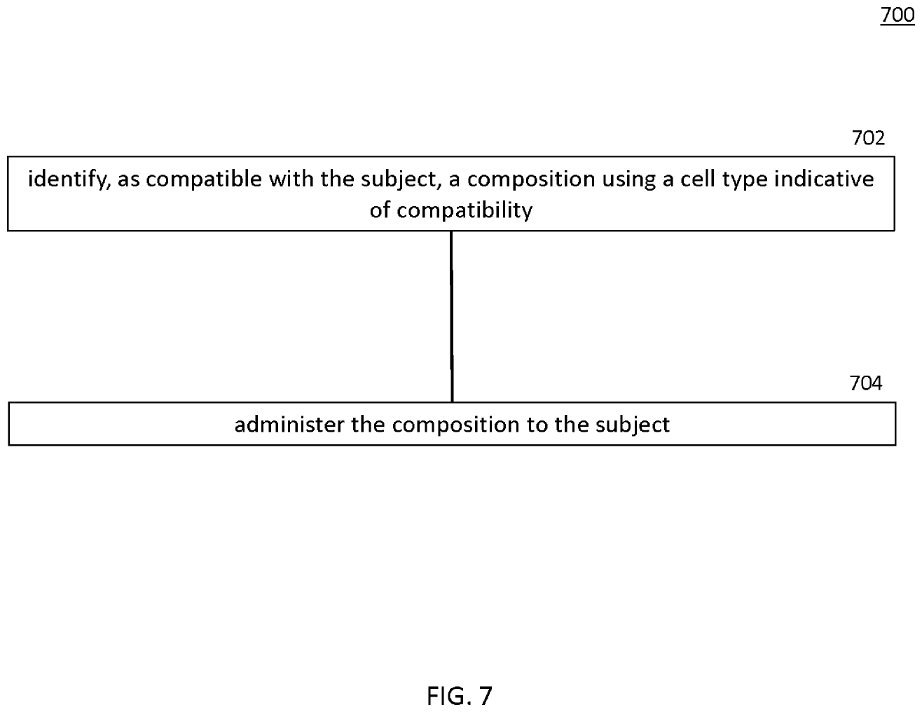

FIG. 7 is a block diagram showing a method 700 of treating a condition in a subject, according to an illustrative embodiment of the invention. In one step 702, an iPSC-derived chondrogenic composition is identified as compatible (e.g., most compatible) with the subject using a cell type indicative of compatibility (e.g., by determining that the HLA loci, associated with the cell(s) from which the iPSC-derived chondrogenic composition is derived are identical to the HLA loci of the subject). The identified iPSC-derived chondrogenic composition is then administered (704) to the subject.

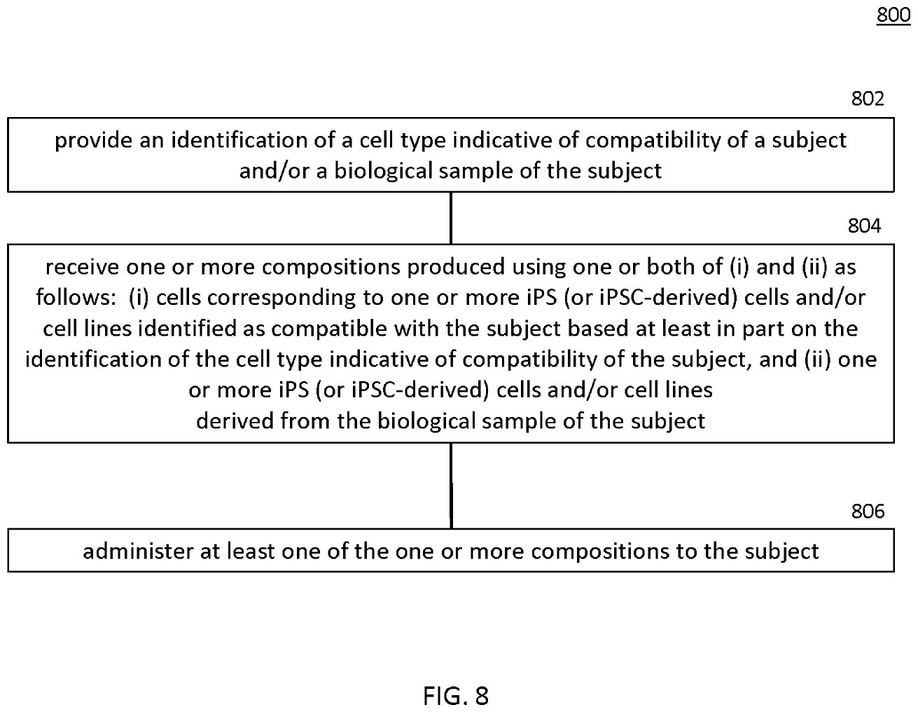

FIG. 8 is a block diagram showing a method 800 of administering an iPSC-derived chondrogenic composition to a subject, according to an illustrative embodiment of the invention. In one step 802, an identification of a cell type indicative of compatibility of a subject and/or a biological sample of the subject is provided. In a following step 804, one or more iPSC-derived chondrogenic compositions produced using one or both of (i) and (ii) as follows: (i) cells corresponding to one or more iPS cells and/or cells lines, and/or iPSC-derived cells and/or cell lines identified as compatible with the subject based at least in part on the identification of the cell type indicative of compatibility of the subject, and (ii) iPS cells and/or cell lines, and/or iPSC-derived cells and/or cell lines derived from the biological sample of the subject is received (e.g., from storage (e.g., in the physical repository)). Then at least one of the received one or more iPSC-derived chondrogenic compositions is then administered (806) to the subject. In certain embodiments, a combination of the one or more iPSC-derived chondrogenic compositions may be administered to the subject.

Generation and Differentiation Protocols for Immortalized iPSCs

Induced pluripotent stem cell (iPSC) generation protocols are described, for example, on the world wide web at hypertext transfer protocol thermofisher.com/us/en/home/references/protocols/cell-culture/stem-cell-protocols/ipsc-protocols.html, the contents of which is hereby incorporated by reference in its entirety. Induced pluripotent stem cell (iPSC) generation and differentiation protocols are described, for example, on the world wide web at hypertext transfer protocol sigmaaldrich.com/life-science/stem-cell-biology/ipsc/ipsc-protocols.html, the contents of which is hereby incorporated by reference in its entirety. Differentiation of iPSCs can be found, for example, in “Induction of Pluripotent Stem Cells from Adult Human Fibroblasts by Defined Factors”; Takahashi K., Tanabe K., Ohnuki M., Narita M., Ichisaka T., Tomoda K., Yamanaka S.; Cell Vol. 131, 861-872, November 2007″, the contents of which is hereby incorporated by reference in its entirety.

Recently, HSCs have been successfully produced from iPSCs. See, for example, “Generation of engraftable hematopoietic stem cells from induced pluripotent stem cells by way of teratoma formation,” Mol Ther. 2013 Jul; 21(7); 1424-31; Epub May 14, 2013; “Hematopoietic stem cells meet induced pluripotent stem cells technology,” Haematologica, 2016 Sep; 101(9): 999-1001; and “In vivo generation of transplantable human hematopoietic cells from induced pluripotent stem cells,” Blood, 2013 Feb 21; 121(8); 1255-64; Epub Dec. 4 2012; the contents of each of which are incorporated herein by reference. Furthermore, in recent years, there have been significant advances in the production of iPSCs from cells collected from a biological sample of a subject (e.g., blood cells). For example, iPSCs can be made by inserting copies of stem cell-associated genes—e.g., Oct 3/4, Sox 2, Klf4, and c-Myc (or Oct 3/4, Sox 2, Nanog, and Lin28)—into cells collected from the biological sample using viral vectors. See, for example, K. Okita, T. Ichisaka, and S. Yamanaka, “Generation of germline-competent induced pluripotent stem cells,” Nature, vol. 448, no. 7151, pp. 313-317, 2007; K. Okita, Y. Matsumura, Y. Sato et al., “A more efficient method to generate integration-free human iPS cells,” Nature Methods, vol. 8, no. 5, pp. 409-412, 2011; the contents of each of which are incorporate herein by reference.

Storage of Immortalized iPSCs

Induced human pluripotent stem cells (iPSCs) can be generated from biological samples, such as blood samples. Depending on the conditions, in vitro iPSCs can retain their pluripotency or they can be directed to differentiate into a wide range of specialized cell types and tissues. Such cell types and tissues can be used for applications including replacement of diseased or damaged tissues in patients with conditions such as trauma, diabetes, degenerative neurological disorders, cardiovascular disease, and metabolic deficiencies.

Repositories (290) (e.g., cell repositories, e.g., nucleic acid repositories) for storing biological sample material (e.g., cells, e.g., nucleic acids) can include liquid nitrogen storage tanks and/or other freezer systems. Liquid nitrogen tanks provide temperature (e.g., about −195° C.) and/or humidity control, and can be used to store, for example, immortalized cell lines (e.g., immortalized iPSCs) over a long period of time. Alternatively, biological material (e.g., nucleic acids) can be stored in freezer systems at higher temperatures (e.g., from about −80° C. to about −20° C.). Additional equipment, backup systems, software/inventory control systems, sample location systems, automated sample retrieval, etc. can be used for storage and/or maintenance of the biological sample material stored in the repositories. The described setup allows for backup systems (e.g., additional repositories) to be used if a given tank and/or freezer temperature control system and/or humidity control system malfunctions.

Moreover, the provided systems and methods can record and track, via a graphical user interface, biological samples (and biological material extracted therefrom) used to generate genotyping data, for example, as described in U.S. Application No. 62/485,778, entitled “Chain Of Custody For Biological Samples And Biological Material Used In Genotyping Tests” and filed on Apr. 14, 2017, U.S. application Ser. No. 15/846, 659 entitled “Chain Of Custody For Biological Samples And Biological Material Used In Genotyping Tests” filed on Dec. 19, 2017, and International Application No. PCT/US17/67272 entitled “Chain of Custody for Biological Samples and Biological Material Used in Genotyping Tests” filed on Dec. 19, 2017, the contents of which are hereby incorporated by reference in their entirety.

For example, as biological samples are processed in several stages to extract biological material and perform genotyping tests, IDs are assigned to biological sample material for individuals as well as well plates used during processing of the biological sample material in order to organize the samples and the tests. Biological sample materials are assigned to well plates for use in extracting biological material. Biological sample material is assigned to genotyping plates for use in performing genotyping tests. By associating IDs corresponding to biological sample material with IDs for well plates or genotyping plates, respectively, a user can track which extractions and/or tests need to be performed as well as record which biological samples have been received or genotyping plates analyzed via a graphical user interface.

Method of Preparing Storable iPSC-Derived Chondrons from iPSC-Derived Chondrocytes:

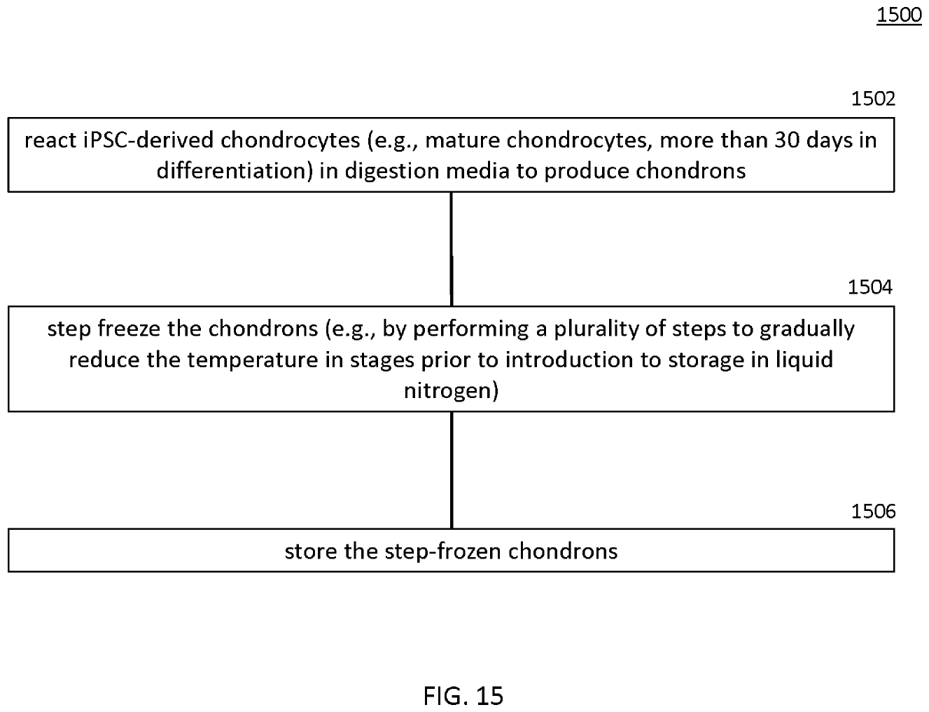

FIG. 15 is a block diagram showing a method 1500 of preparing storable iPSC-derived chondrons from iPSC-derived chondrocytes, according to an illustrative embodiment of the invention. In one step 1502, iPSC-derived chondrocytes (e.g., mature chondrocytes, more than 30 days in differentiation) are reacted in digestion media to produce iPSC-derived chondrons. In a following step 1504, iPSC-derived chondrons are frozen by step freezing (e.g., by performing a plurality of steps to gradually reduce the temperature in stages prior to introduction to storage in liquid nitrogen). In certain embodiments, for example, a step freezing protocol comprises the steps of (i) freezing for time t₁ min at temperature T₁° C., followed by (ii) freezing for time t₂ min at temperature T₂° C. (e.g., T₁ is higher than T₂) and then optionally (iii) repeating steps (i) and (ii) until desired temperature and/or freezing is achieved. Then in step 1506, the frozen iPSC-derived chondrons are stored (e.g., in a biorepository). In certain embodiments, the frozen iPSC-derived chondrons are stored in liquid nitrogen at −195° C.

Elements of embodiments involving one aspect of the invention can be applied in embodiments involving other aspects of the invention.

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

### Illustrative Computer Network Environment

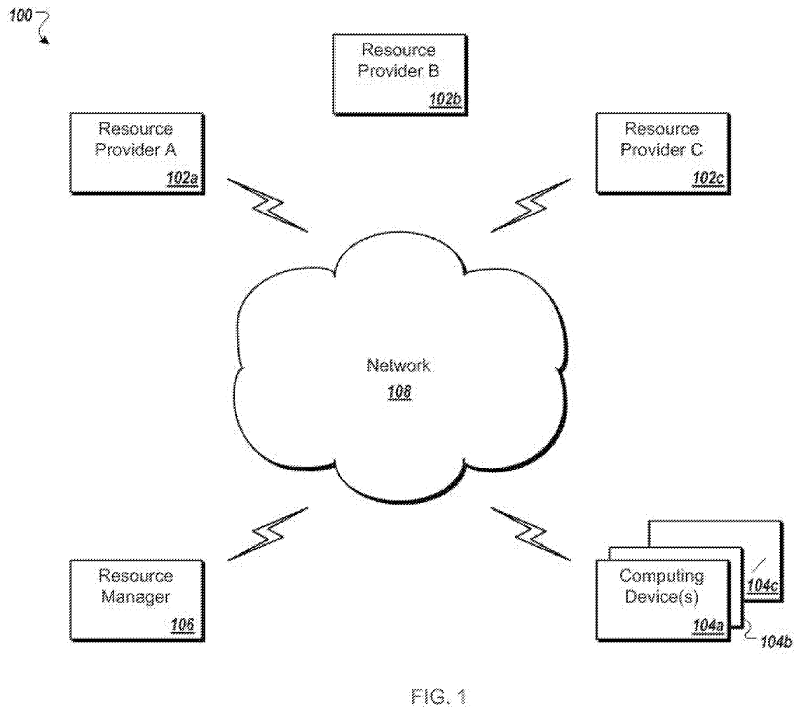

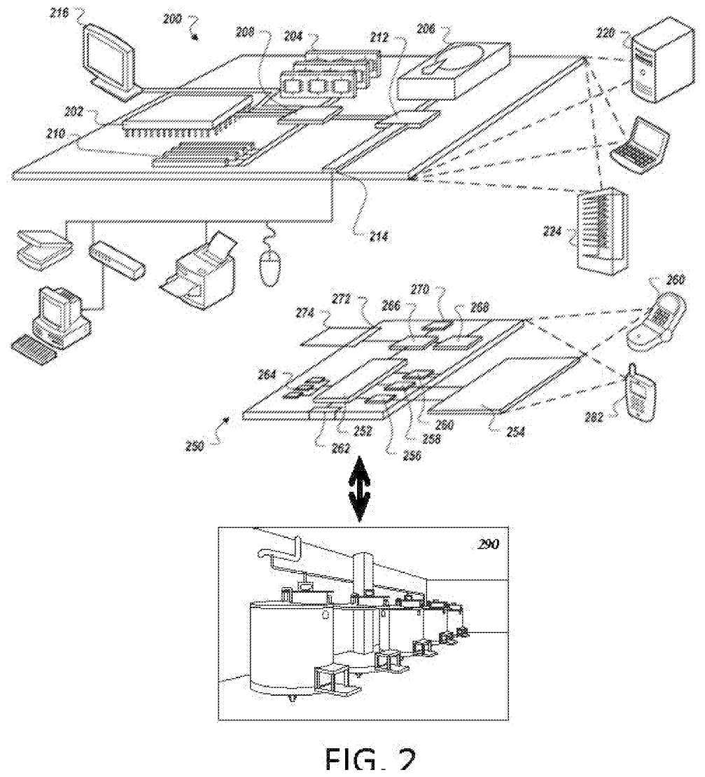

FIG. 1 shows an illustrative network environment 100 for use in the methods and systems described herein. In brief overview, referring now to FIG. 1, a block diagram of an exemplary cloud computing environment 100 is shown and described. The cloud computing environment 100 may include one or more resource providers 102a, 102b, 102c (collectively, 102). Each resource provider 102 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 102 may be connected to any other resource provider 102 in the cloud computing environment 100. In some implementations, the resource providers 102 may be connected over a computer network 108. Each resource provider 102 may be connected to one or more computing device 104a, 104b, 104c (collectively, 104), over the computer network 108.

The cloud computing environment 100 may include a resource manager 106. The resource manager 106 may be connected to the resource providers 102 and the computing devices 104 over the computer network 108. In some implementations, the resource manager 106 may facilitate the provision of computing resources by one or more resource providers 102 to one or more computing devices 104. The resource manager 106 may receive a request for a computing resource from a particular computing device 104. The resource manager 106 may identify one or more resource providers 102 capable of providing the computing resource requested by the computing device 104. The resource manager 106 may select a resource provider 102 to provide the computing resource. The resource manager 106 may facilitate a connection between the resource provider 102 and a particular computing device 104. In some implementations, the resource manager 106 may establish a connection between a particular resource provider 102 and a particular computing device 104. In some implementations, the resource manager 106 may redirect a particular computing device 104 to a particular resource provider 102 with the requested computing resource.

FIG. 2 shows an example of a computing device 200 and a mobile computing device 250 that can be used in the methods and systems described in this disclosure. The computing device 200 is intended to represent various forms of digital computers, such as laptops, desktops, workstations, personal digital assistants, servers, blade servers, mainframes, and other appropriate computers. The mobile computing device 250 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 200 includes a processor 202, a memory 204, a storage device 206, a high-speed interface 208 connecting to the memory 204 and multiple high-speed expansion ports 210, and a low-speed interface 212 connecting to a low-speed expansion port 214 and the storage device 206. Each of the processor 202, the memory 204, the storage device 206, the high-speed interface 208, the high-speed expansion ports 210, and the low-speed interface 212, are interconnected using various busses, and may be mounted on a common motherboard or in other manners as appropriate. The processor 202 can process instructions for execution within the computing device 200, including instructions stored in the memory 204 or on the storage device 206 to display graphical information for a GUI on an external input/output device, such as a display 216 coupled to the high-speed interface 208. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 204 stores information within the computing device 200. In some implementations, the memory 204 is a volatile memory unit or units. In some implementations, the memory 204 is a non-volatile memory unit or units. The memory 204 may also be another form of computer-readable medium, such as a magnetic or optical disk.

The storage device 206 is capable of providing mass storage for the computing device 200. In some implementations, the storage device 206 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 202), perform one or more methods, such as those described above. The instructions can also be stored by one or more storage devices such as computer- or machine-readable mediums (for example, the memory 204, the storage device 206, or memory on the processor 202).

The high-speed interface 208 manages bandwidth-intensive operations for the computing device 200, while the low-speed interface 212 manages lower bandwidth-intensive operations. Such allocation of functions is an example only. In some implementations, the high-speed interface 208 is coupled to the memory 204, the display 216 (e.g., through a graphics processor or accelerator), and to the high-speed expansion ports 210, which may accept various expansion cards (not shown). In the implementation, the low-speed interface 212 is coupled to the storage device 206 and the low-speed expansion port 214. The low-speed expansion port 214, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 200 may be implemented in a number of different forms, as shown in the figure. For example, it may be implemented as a standard server 220, or multiple times in a group of such servers. In addition, it may be implemented in a personal computer such as a laptop computer 222. It may also be implemented as part of a rack server system 224. Alternatively, components from the computing device 200 may be combined with other components in a mobile device (not shown), such as a mobile computing device 250. Each of such devices may contain one or more of the computing device 200 and the mobile computing device 250, and an entire system may be made up of multiple computing devices communicating with each other.

The mobile computing device 250 includes a processor 252, a memory 264, an input/output device such as a display 254, a communication interface 266, and a transceiver 268, among other components. The mobile computing device 250 may also be provided with a storage device, such as a micro-drive or other device, to provide additional storage. Each of the processor 252, the memory 264, the display 254, the communication interface 266, and the transceiver 268, are interconnected using various buses, and several of the components may be mounted on a common motherboard or in other manners as appropriate.

The processor 252 can execute instructions within the mobile computing device 250, including instructions stored in the memory 264. The processor 252 may be implemented as a chipset of chips that include separate and multiple analog and digital processors. The processor 252 may provide, for example, for coordination of the other components of the mobile computing device 250, such as control of user interfaces, applications run by the mobile computing device 250, and wireless communication by the mobile computing device 250.

The processor 252 may communicate with a user through a control interface 258 and a display interface 256 coupled to the display 254. The display 254 may be, for example, a TFT (Thin-Film-Transistor Liquid Crystal Display) display or an OLED (Organic Light Emitting Diode) display, or other appropriate display technology. The display interface 256 may comprise appropriate circuitry for driving the display 254 to present graphical and other information to a user. The control interface 258 may receive commands from a user and convert them for submission to the processor 252. In addition, an external interface 262 may provide communication with the processor 252, so as to enable near area communication of the mobile computing device 250 with other devices. The external interface 262 may provide, for example, for wired communication in some implementations, or for wireless communication in other implementations, and multiple interfaces may also be used.

The memory 264 stores information within the mobile computing device 250. The memory 264 can be implemented as one or more of a computer-readable medium or media, a volatile memory unit or units, or a non-volatile memory unit or units. An expansion memory 274 may also be provided and connected to the mobile computing device 250 through an expansion interface 272, which may include, for example, a SIMM (Single In Line Memory Module) card interface. The expansion memory 274 may provide extra storage space for the mobile computing device 250, or may also store applications or other information for the mobile computing device 250. Specifically, the expansion memory 274 may include instructions to carry out or supplement the processes described above, and may include secure information also. Thus, for example, the expansion memory 274 may be provided as a security module for the mobile computing device 250, and may be programmed with instructions that permit secure use of the mobile computing device 250. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 252), perform one or more methods, such as those described above. The instructions can also be stored by one or more storage devices, such as one or more computer- or machine-readable mediums (for example, the memory 264, the expansion memory 274, or memory on the processor 252). In some implementations, the instructions can be received in a propagated signal, for example, over the transceiver 268 or the external interface 262.

The mobile computing device 250 may communicate wirelessly through the communication interface 266, which may include digital signal processing circuitry where necessary. The communication interface 266 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 268 using a radio-frequency. In addition, short-range communication may occur, such as using a Bluetooth®, Wi-Fi™, or other such transceiver (not shown). In addition, a GPS (Global Positioning System) receiver module 270 may provide additional navigation- and location-related wireless data to the mobile computing device 250, which may be used as appropriate by applications running on the mobile computing device 250.

The mobile computing device 250 may also communicate audibly using an audio codec 260, which may receive spoken information from a user and convert it to usable digital information. The audio codec 260 may likewise generate audible sound for a user, such as through a speaker, e.g., in a handset of the mobile computing device 250. Such sound may include sound from voice telephone calls, may include recorded sound (e.g., voice messages, music files, etc.), and may also include sound generated by applications operating on the mobile computing device 250.

The mobile computing device 250 may be implemented in a number of different forms, as shown in the figure. For example, it may be implemented as a cellular telephone 280. It may also be implemented as part of a smart-phone 282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications, or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In certain embodiments, the system comprises a physical biorepository 290(comprising one or more cell storage containers) in communication with any of the computer system arrangements of FIGS. 1 or 2.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

It is contemplated that compositions, methods, and systems of the claimed subject matter encompass variations and adaptations developed using information from the embodiments described herein. Adaptation and/or modification of the compositions, methods, and systems described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

Elements of embodiments described with respect to a given aspect of the claimed subject matter may be used in various embodiments of another aspect of the claimed subject matter. For example, it is contemplated that features of dependent claims depending from one independent claim can be used in compositions, systems, and/or methods of any of the other independent claims.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting with respect to the claimed subject matter.

Documents are incorporated herein by reference as noted. Where there is any discrepancy in the meaning of a particular term, the meaning provided in the Definition section above is controlling.

Certain embodiments of the present invention are described herein. It is, however, expressly noted that the present invention is not limited to these embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description. Therefore, the disclosure should not be limited to certain implementations, but rather should be limited only by the spirit and scope of the following claims.

### EXEMPLIFICATION

### Example 1

### Chondrocyte Differentiation

Differentiation of iPSC-derived Mesenchymal Stems Cells (MSCs) to chondrocytes was performed.

A single cell suspension of Mesenchymal Stems Cells (MSCs) was prepared using the cell detachment solution ACCUTASE. Any further dissociation was prevented using MSC 37° C. culture media. The harvested cells were centrifuged in a bench top centrifuge at 500 * g for 5 minutes. The cells were then washed with 1× High Glucose (HG) Dulbecco's Modified Eagle Medium (DMEM), a basal medium for supporting growth of many different mammalian cells. The cells were resuspended at a density of 1.25 * 10⁶ cells/ml in chondrogenic differentiation medium (see Table 1). Then, 0.2 ml aliquots of cell suspension (2.5×10⁵ cells/well) were dispensed into the 96 wells of a U-bottomed well plate. The plate was then centrifuged at 500 * g for 5 minutes, following which they were placed in an incubator at 37° C., 5% CO₂. Following 24 hours of cell plating in the 96-well plates, the pellet was released from the bottom of the wells by aspirating 100 μl of media and gently releasing the media back into the wells using an eight-channel pipette. The chondrogenic differentiation media was replaced every other day. Full differentiation was observed 30 days from the addition of chondrogenic differentiation media, based on the expression of the proteoglycan called aggrecan and Collagen II markers.

TABLE 1
Recipe for Chondrogenic Differentiation Media

Component

| Dulbecco's Modified Eagle's Medium, High | 4.5 g/l High Glucose | |
|---|---|---|
| Glucose (DMEM-HG) | | |
| ITS (Insulin-Transferrin-Selenium solution, | 1% v/v | |
| Gibco) + Premix Tissue Culture Supplement | | |
| (Becton Dickinson) | | |
| Dexamethasone | 100 | nM |
| Ascorbate-2-Phosphate | 200 | μM |
| Sodium Pyruvate | 1% | |
| Transforming Growth Factor-Beta 1 (TGF-β1) | 10 | ng/ml |

| | |
|---|---|
| Penicillin / Streptomycin | 1× |

### Example 2

### iPSC-Derived Chondrocyte Freezing and Thawing

iPSC-derived chondrocyte freezing and thawing protocols that produce viable chondrocytes was performed.

For freezing the cultured chondrocytes, each chondrocyte cell block was transferred without breaking up the block while transferring from the U-bottomed 96-well plate to a 1.8 ml cryotube using a P1000 tip. Each cryotube was filled with 10-15 cell blocks. Any excess differentiation media was removed, leaving only 50 μl to cover the cell blocks during freezing. Then 1 ml freezing media (StemCell Banker, AMSBIO—Nippon Zenyaku Kogyo) was added to each cryotube, and the suspension was mixed gently using a P1000 tip. The cryotubes were transferred to 4° C. for 4 hours and then placed into chilled CoolCell (BioCision) alcohol-free cell freezing container. The cryotubes in the CoolCell were then transferred to −80° C. for overnight storage and finally stored in liquid nitrogen at 195° C.

For thawing the frozen chondrocytes, each cryotube was warmed in a warm bath maintained at 37° C. until a few ice crystals were remaining. Then, under sterile conditions, the contents of the cryotube were transferred into a 15 ml conical tube with 9 ml DMEM media pre-warmed to 37° C. The conical tubes were then centrifuged at 1000 rpm for 3 minutes. The chondrocyte pellets were then resuspended directly into chondrogenic differentiation media.

### Example 3

### Assessment of iPSC-Derived Chondrogenic Composition in Animal Model

A single dose study of the efficacy of chondrogenic compositions comprising chondrons, which are mature chondrocytes together with their pericellular matrix, in a rat medial meniscal tear (MMT) model was performed.

Cell Preparation: Experimental chondrocytes (G3N-Chond_001) were shipped as non-adherent cell blocks or chondrons in 25 ml T-flask. The excess chondrocyte culture media in the 25 ml T-flask was removed and discarded. Then, under sterile conditions, 15 ml of fresh chondrocyte culture media was added and the cells were stored in a cell culture incubator at 37° C. and 5% CO₂ until use. The stored cells were found to be stable for up to one week.

Cell Isolation: On the day of the experiment, the non-adherent cell blocks (chondrons) stored in the cell culture incubator were transferred to a 50 ml conical tube. The conical tube was centrifuged at 150 × g for 5 minutes at room temperature. Then, 15 ml of 1× Phosphate Buffered Saline (PBS) was added to the conical tube, and the tube was centrifuged a second time at 150 × g for 5 minutes at room temperature. The pellet was re-suspended in 2 ml of collagenase working solution pre-warmed to 37° C. The conical tube was then incubated at 37° C. and 5% CO₂ for 60 minutes. The cap of the conical tube was loosened while inside the incubator and the solution was mixed every 10 minutes. At the end of the 60 minute incubation time, the cell blocks or chondrons where slowly passed through a 1 ml pipette 5 times so as to disassociate the cell blocks into smaller clumps of chondrons and/or individual cells. The reaction was stopped by adding 15 ml of the chondrocyte culture media. Following this, the chondron-chondrocyte cell suspension was centrifuged at 200 × g for 5 minutes at room temperature. The pellet was then re-suspended in 5 ml of chondrocyte culture media and a cell count was performed. Further, the percent viability and the total number of cells were also determined. The cell suspension was then centrifuged at 200 × g for 5 minutes at room temperature, following which the cells were re-suspended in HG-DMEM solution at a concentration of 1×10⁶ cells/ml.

Animal Model: The rat medial meniscal tear (MMT) model was used to assess the potential of the chondrogenic compositions. Animals were anesthetized with Isoflurane and the right knee area and lower leg were prepared for surgery. A skin incision was made from the distal patella to the proximal tibial plateau. The medial collateral ligament was transected just below its attachment to the meniscus, so that when the joint space opened, the meniscus was reflected toward the femur. The meniscus was cut at its narrowest point (away from the ossicles), and care was taken not to damage the tibial surface and to ensure the resulting transection yielded a meniscus that is freely movable both anteriorly and posteriorly.

Disease Parameters and Progression: Cartilage degenerative changes develop progressively and, by 3 to 6 weeks post-surgery, tibial cartilage degeneration may be focally severe on the outer ⅓ of the tibia, with degenerative changes of lesser severity in the middle and inner ⅓. A group size of 15 to 20 animals is generally sufficient to account for variable lesion severity. Because cartilage degenerates rapidly in rats, this model constitutes an extremely high hurdle; however, zonal analysis may result in detection of treatment effects in the middle and inner one-third of the tibia, areas in which mechanical trauma is not an important cause of pathogenesis. This model therefore permits evaluation not only of chondro-protective effects, but also bone preserving activities, as well as synovium as a measure of inflammation.

Dosing Regimen: The treatment dosing was started one week post-surgery and a necropsy was performed at 3 weeks. Fifty thousand cells were injected as a single dose for each animal. The route of administration was into the knee joint on one side of the animal.

Clinical Assessment: Animal body weights were measured weekly. Tactile allodynia and Gait analysis were also evaluated at multiple time-points.

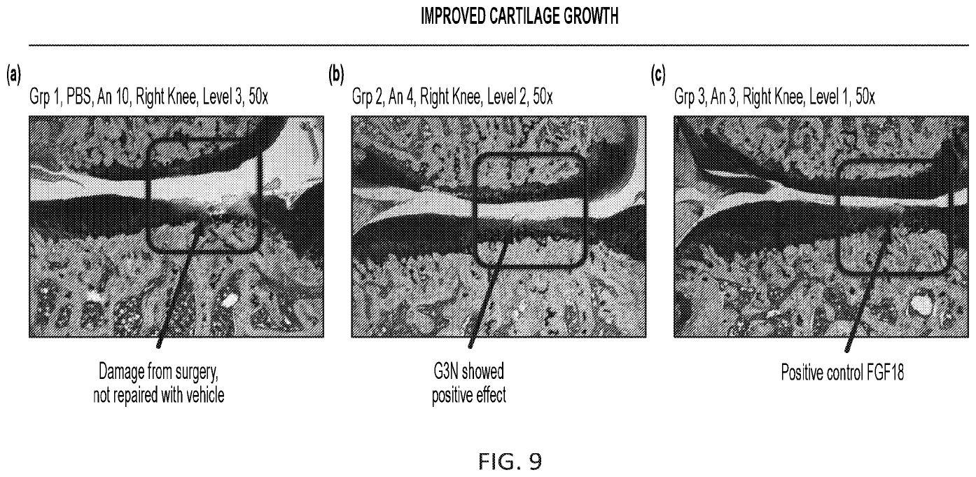

Results: Operated joints were cut into two approximately equal halves in the frontal plane and embedded in paraffin. Three sections were cut from each knee at approximately 160 μm steps and stained with toluidine blue. The stained tissues were then analyzed microscopically. The values for each parameter were then averaged across the three sections to determine overall values for each animal. Knees were examined microscopically by a board certified veterinary pathologist according to these methods. FIG. 9 presents the histological analysis of the toluidine stained sections. The left image (a) shows the damage from the surgery in the vehicle treated animal. As observed from this representative image, animals treated with the vehicle on average showed no repair of the damage from surgery. The middle image (b) shows the repair of the cartilage after treatment with the chondrogenic composition. As observed from this image, animals treated with the chondrogenic composition show significant improvement and repair of the damage to the knee. This improvement and repair is similar to the repair observed in animals treated with FGF18 positive control. The right image (c) demonstrates repair of the cartilage after treatment with FGF18 which stimulates chondrogenesis.

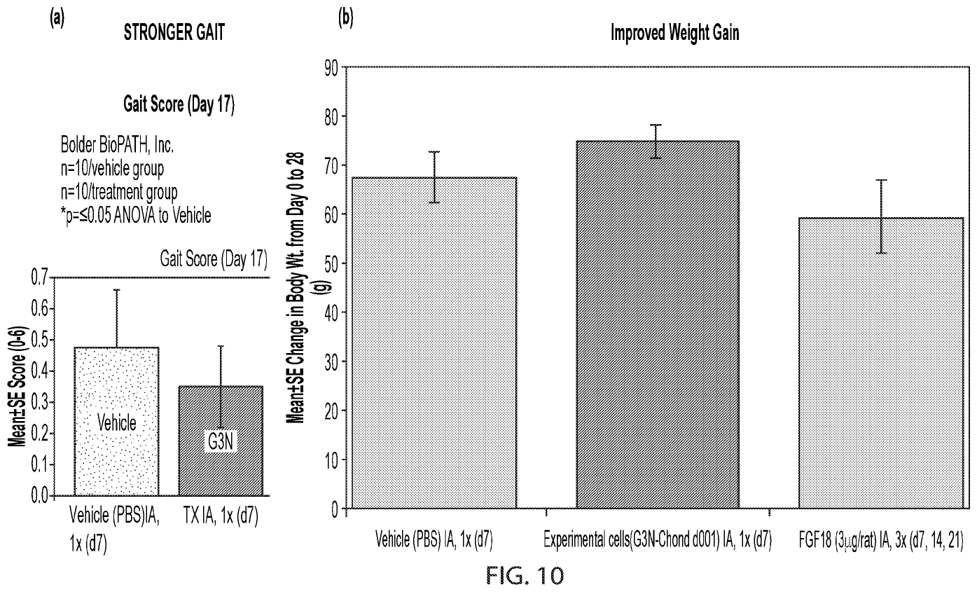

Gait and weight analyses were also performed on the rats throughout the duration of the experiment. FIG. 10 presents the results of these studies at the end of the experiment. The left figure (a) demonstrates changes from the gait analysis using a gait scoring system. Animals that were treated with the chondrogenic composition were observed to have better gaits, in other words lower gait scores, compared to the vehicle control. Weight analysis of the animals also showed that the animals treated with the chondrogenic composition (FIG. 10 (b) (right image)) showed improved weight gain compared to vehicle or the positive control.

### Example 4

### Differentiation of iPSCs into MSCs (“iMSCs” or “ORIG3N-MSCs”)

Differentiation of iPSCs in iPSC-derived Mesenchymal Stems Cells (MSCs) was performed.

Preparation of fibrillar Type I collagen coatings: Purified bovine Type I collagen monomer was stabilized in an acidic solution (3 mg/ml) (PureCol, Inamed Biomaterials, CA) and was neutralized using NaOH in PBS. The solution was mixed in an 50 ml conical tube and in following order : 35 ml PBS+0.5 ml 0.1N NaOH+0.5 ml (10×) PBS+4m1 Purecol.

One ml of this diluted solution was added per well in 12-well non-tissue culture plates. After incubation at 37° C. in an incubator with 5% CO₂ for 16-21 hours, the gel-like supernatant was aspirated from the plates and the coated surfaces were rinsed with water and PBS. 1 ml of culture media was added and the surface was kept moist before adding cells.

If needed, this coated plate can be prepared in advance, e.g., by air drying coated collagen in the hood and keeping the coated plate at 4° C. The surface is hydrated for at least 2 h before adding cells.

Differentiation of iPSCs into iMSCs: DAY 0: Before dissociation, iPSCs were treated with 10 μM ROCK inhibitor Y-27632 in maintenance medium for 1 hour. Colonies were then dissociated into single cells after incubating 2-3 min with ACCUTASE. Single cells were seeded onto the collagen coated well at a density of 15,000/cm² in the iPSCs maintenance medium (TeSR E8 complete).

DAY 1: iPSC maintenance medium was supplemented with an equal volume of the Differentiation Medium which contained basal alpha-MEM (GIBCO), 10% human AB serum (SIGMA), 100 U/ml penicillin and 100 μg/m1 streptomycin (GIBCO), 100 nM dexamethasone (Sigma-Aldrich) and 50 μM magnesium L-ascorbic acid phosphate (Sigma-Aldrich).

DAY 3: Full medium change with the differentiation medium. After this, medium was changed every 3-4 days.

DAY 10: Cells were harvested and labeled as passage zero (P0). Half were used for 1st frozen stock. Remaining cells were expanded on new collagen coating using Expansion Medium: alpha-MEM (GIBCO), 10% Human AB serum (SIGMA) , 100 U/ml penicillin and 100 pg/m1 streptomycin, 2 mM L-glutamine, 0.1 mM non-essential amino acid (all from Invitrogen) and GSK3 inhibitor CHIR99021 (TOCRIS) at a final concentration of 3 μM.

The expansion medium was replenished every 3-4 days. Cells were passaged upon subconfluency, at a 1:3 split ratio. The second passage (P2) was used for the tri-lineage differentiation evaluation and flow cytometry analysis.

Flow cytometry markers included the following surface markers: CD73, CD90, CD105, CD34 and CD45.

Differentiation of these iMSCs into, for example, chondrocytes, adipocytes, and osteoclasts, has been demonstrated by the Applicant.

### Example 5

### Method for Transcriptome Analysis of iPSC-Derived MSCs

Transcriptome analyses of iPSC-derived MSCs and Bone Marrow-derived MSCs (BM-MSCs) were performed and compared.

Samples: RNA expression analysis was performed on iPSC-derived MSCs (ORIG3N-MSCs) derived from two independent preparations and compared with the RNA expression of commercially purchased Bone Marrow (BM) derived-MSCs (Promocell, cat.# C14090) and cord blood derived CD34 positive mononuclear cells (Lonza, cat.#2C-101B).

RNA preparation and characterization: For total RNA preparation, the NucleoSpin RNA plus Kit (Macheray-Nagel, ref # 740984.50) was used. This kit allowed the removal of genomic DNA without the need for enzymatic treatment, thereby producing high quality RNA for sensitive downstream applications. Concentration and quality of the purified RNA was assessed using Nanodrop 8000 (FisherScientific) and the RNA Integrity Number (RIN) was determined using a Bioanalyzer at Boston Children's Hospital Core Facility. Only samples with high RIN number were used for the RNA sequencing analysis.

RNA Sequencing and Transcriptome analysis: For the differential characterization of the transcriptomes between the different sources of cells we use Ion AmpliSeg™ Transcriptome Human Gene Expression Panel, Chef-Ready Kit (ThermoFisher, Cat.#A31446). This method allowed for the simultaneous gene expression measurement of over 20,000 human reference genes (RefSeq) in a single assay using a small amount of RNA (10 ng Total RNA). Specific amplicons were sequenced and analyzed using Ion-Proton Sequencing System using Torrent Suite Software. The results reported in Table 2 were normalized as reads per million, which may be directly used for comparing the gene expression level across samples. The results were normalized to Reads per Million (RPM) and reported as log₁₀ values of the normalized RPMs. For example, a value of 3 RPM for the transcript of the CXCL14 gene in the iPSC-derived MSC sample corresponds to 1000 read counts (or copies) per million total reads of the sample. A value of 0 RPM for a particular gene (for example CXCR4 in the BM-derived MSC) corresponds to no read counts (or copies) in the sample. As observed from Table 2, the number of transcripts of each of the genes (listed in column 2 of Table 2) were determined to be higher in the iPSC-derived MSCs than the BM-derived MSCs, suggestive of the better therapeutic potential of iPSC-derived MSCs.

TABLE 2
Characterization of transcriptome differences between
human Bone Marrow MSCs (BM-MSC) and iPSC-derived
MSCs (ORIG3N-MSCs) according to multiplex transcriptome
analysis (AmpliSeq). Values as Log₁₀ Reads per
Million (RPM). 0 = no reads.

| Chemokynes and | | | |
|---|---|---|---|
| Chemokyne | | | iPSC-derived |
| Receptors | Gene | BM-derived MSC | MSC |
| Inflammatory | CXCL1 | 2.22 | −1 |
| pathways | CXCL12 | 0.84 | 1.41 |
| | CXCL14 | −0.70 | 3 |
| | CXCR4 | 0 | 1 |
| | CXCR7 | 0 | 1 |
| | CCL2 | 1.25 | 2 |
| | CCL5 (RANTES) | 0 | 1 |
| | IDO1 | 0 | 1.25 |
| | A2M | 0 | 1.30 |
| Important for | EGFL6 | 0 | 2.46 |
| chondrogenesis | DCN | 2.51 | 3.37 |
| | BMP2 | 0 | 1.27 |
| | BMPR1A | 1.44 | 1.77 |
| | BMP4 | 0 | 1.89 |
| | BMPR1B | 0 | 1.30 |
| | BMPR2 | 2.03 | 2.22 |
| | IGF2 | 0 | 2 |
| Collagens | LRP5 | 1.4 | 1.8 |
| | LRP6 | 1.8 | 2.18 |
| | VCAM1 | 1 | 2 |
| | VCAN | 2 | 3 |
| | CILP2 | 0 | 3 |
| | COL10A1 | 2 | 0 |
| | COL2A1 | 0 | 2 |
| | COLL3A1 | 3 | 4 |
| | COL4A1 | 2 | 3 |
| | COL4A2 | 2 | 3 |
| Proteoglycan | AGRN | 1 | 2.4 |

### Example 6

### Assessment of iPSC-Derived Chondrocyte and iPSC-Derived MSC Compositions in Animal Model

A dose study of the efficacy of various iPSC-derived compositions in a rat medial meniscal tear (MMT) model was performed. Compositions comprising one or more of iPSC-derived MSCs, and iPSC-derived chondrocytes were compared to BM-derived MSCs, and positive and negative controls. The compositions comprising iPSC-derived chondrocytes were prepared from frozen iPSC-derived chondrons; as noted previously, iPSC-derived chondrons are mature iPSC-derived chondrocytes together with their pericellular matrix (PCM).

Animal Model: The rat medial meniscal tear (MMT) model was used to assess the potential of the compositions. Animals were anesthetized with Isoflurane and the right knee area and lower leg prepared for surgery. A skin incision was made from the distal patella to the proximal tibial plateau. The medial collateral ligament was transected just below its attachment to the meniscus, so that when the joint space was opened, the meniscus was reflected toward the femur. The meniscus was cut at its narrowest point (away from the ossicles), and care was taken not to damage the tibial surface and to ensure the resulting transection yielded a meniscus that was freely movable both anteriorly and posteriorly. The damage produced by this surgery reproduces the pattern and progression of cartilage degeneration in a controlled fashion so that symptoms and repair may be monitored.

Dosing Regimen: The treatment dosing was started one week (7 days) post-surgery and the composition comprising the iPSC-derived cells (one or more of iPSC-derived MSCs, and iPSC-derived chondrocytes) was injected into the joint via an intra-articular (IA) injection. For controls, compositions comprising BM-derived MSCs, Hyaluronic acid (HA), and PBS were injected into the joints via an intra-articular (IA) injection.

Experimental Study: This study was performed to determine the effects of compositions comprising experimental cells (iPSC-derived chondrocytes, iPSC-derived MSCs, 1:1 mix of iPSC-derived chondrocytes and iPSC-derived MSCs, BM-derived MSCs) and controls (Hyaluronic acid (HA) and PBS). A unit dose of the composition comprising 150,000 cells were delivered via intra-articular (IA) injection one week post-surgery in a rat meniscal tear induced model of osteoarthritis. Male Athymic Nude rats (Hsd:RH-Foxnlrnu) underwent a unilateral medial meniscectomy on Study Day 0. Administration of control Vehicle (PBS) or experimental cells (150 K cells/rat, 50 μl administered via IA) occurred on Study Day 7, and a second injection was given to a subset of animals on Study Day 14. One set of animals were euthanized on Study Day 28 and a second set of animals were euthanized on Study Day 56. See Table 3 for details of experiment.

Assessment: Outcome measures included direct measurements of inflammatory molecules from a lavage of the injured joint and histological measurements of inflammation (synovitis) and cartilage regeneration as a result of the damage. Endpoints for evaluation of therapeutic efficacy included gait analysis on Day 17 and histopathologic examination of chondrocyte death/cartilage degeneration in the knee was initiated at the scheduled time of euthanasia. The joint tissue was processed for histology on Study Day 28 and Study Day 56 after the surgery. Liquid was collected from the joint of each animal via lavage and cytokines were measured on Study Day 28. Blood (for processing into serum) was collected from each animal at the scheduled time of euthanasia. All animals survived to termination.

TABLE 3
Animal groups, corresponding treatments and experimental setup.¹

| | | Compound | Dose | | | | | |
|---|---|---|---|---|---|---|---|---|
| | | And/or | Level | | Dose | Term | Dose | Dose |
| Group | N | Treatment | mg/rat | Route | Regimen | Day | Vol | Conc |
| Grp 1 | 10 | Cell Type #1 | 150K | IA | (d 7) | d 28 | 50 μl | 3M/ml |
| | | iPSC-derived | | | | | | |
| | | MSC | | | | | | |
| Grp 2 | 10 | Cell Type #1 | 150K | IA | (d 7) | d 56 | 50 μl | 3M/ml |
| | | iPSC-derived | | | | | | |
| | | MSC | | | | | | |
| Grp 3 | 10 | Cell Type #2 | 150K | IA | (d 7) | d 28 | 50 μl | 3M/ml |
| | | iPSC-derived | | | | | | |
| | | chondrocytes | | | | | | |
| Grp 4 | 10 | Cell Type #2 | 150K | IA | (d 7) | d 56 | 50 μl | 3M/ml |
| | | iPSC-derived | | | | | | |
| | | chondrocytes | | | | | | |
| Grp 5 | 10 | PBS | N/A | IA | (d 7) | d 28 | 50 μl | N/A |
| Grp 6 | 10 | HA | 0.4 | IA | (d 7) | d 28 | 50 μl | 8 mg/ml |
| | | | mg/knee | | | | | |
| Grp 7 | 10 | Cell Type #3 | 1:1 ratio | IA | (d 7) | d 28 | 50 μl | 3M/ml |
| | | iPSC-derived | | | | | | |
| | | MSC and iPSC- | | | | | | |
| | | derived | | | | | | |
| | | chondrocytes | | | | | | |
| Grp 8 | 10 | Cell Type #3 | 1:1 ratio | IA | (d 7) | d 56 | 50 μl | 3M/ml |
| | | iPSC-derived | | | | | | |
| | | MSC and iPSC- | | | | | | |
| | | derived | | | | | | |
| | | chondrocytes | | | | | | |
| Grp 9 | 10 | commercial BM- | 150K | IA | (d 7) | d 28 | 50 μl | 3M/ml |
| | | MSCs | | | | | | |
| Grp 10 | 10 | commercial BM- | 150K | IA | (d 7) | d 56 | 50 μl | 3M/ml |
| | | MSCs | | | | | | |

¹The compositions of iPSC-derived chondrocytes were prepared by thawing frozen iPSC-derived chondrons.

### Results:

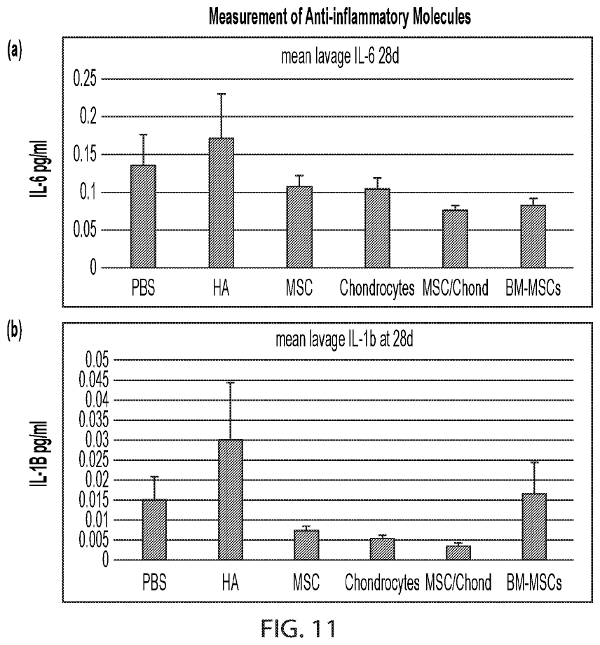

Cytokine Measurements: Cytokines IL-6 and IL-1b were measured from rinsing of the joint and the fluid collected from the joint, also known as lavage. Both the cytokines measured are important mediators of the inflammatory response. These cytokine levels were measured using antibodies and Luminex technology. IL-6 levels have been found to commonly correlate with the severity of injury, while IL-1b levels are known to exacerbate damage during chronic disease and acute injury. Thus, a lower score for both of these suggest a lower inflammatory response. As shown in FIG. 11, the mean lavage shows similar or lower IL-6 and IL-1b cytokine levels in rats administered the iPSC-derived compositions (iPSC-derived MSCs, iPSC-derived chondrocytes, and 1:1 mix of iPSC-derived MSCs and iPSC-derived chondrocytes) compared to the compositions comprising BM-derived MSCs or the controls (HA and PBS).

Synovitis Score: Synovitis is the medical term for inflammation of the synovial membrane. The synovium is the soft tissue that lines the inner surface of the joint and creates the synovial fluid which lubricates the joint and provides some nutrients in the absence of vascularization. The condition of synovitis often occurs from joint damage or disease and causes swelling and is usually very painful when the joint is moved.

Synovial inflammation (mainly mononuclear cell infiltration concentrated on the medial side) was scored as follows. Lower score indicates reduced synovitis. Descriptions of other changes (typically fibrosis, or acute inflammation/neutrophil infiltration extending into the lateral compartment-usually associated with IA treatments) were also scored as listed below:

0=Normal synovium

0.5=Very minimal synovitis (generally focal or scattered minimal diffuse)

1=Minimal synovitis (generally focal or scattered minimal diffuse)

2=Mild synovitis multifocal to confluent areas of mild mononuclear cell infiltration)

3=Moderate synovitis (confluent areas of moderate mononuclear cell infiltration)

4=Marked synovitis (confluent areas of marked mononuclear cell infiltration)

5=Severe synovitis (confluent areas of severe mononuclear cell infiltration)

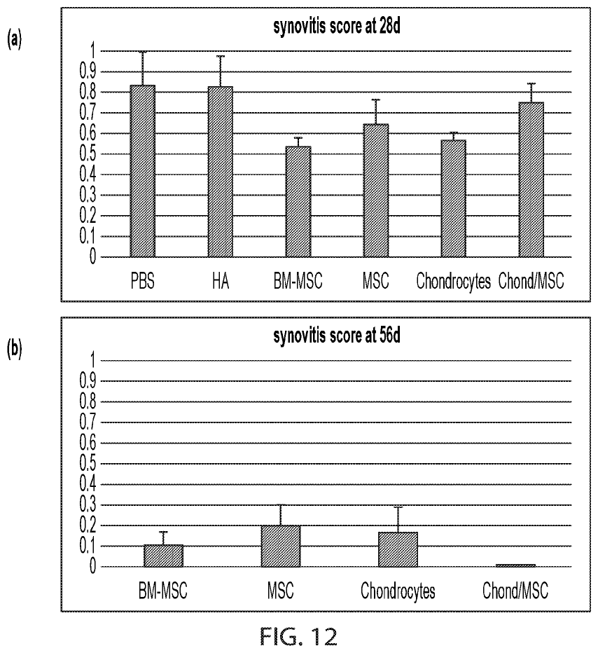

As seen from FIG. 12 synovitis is significantly reduced to the very minimal range (0.5) after cell treatments after 28 days and to an even greater extent to almost normal levels after 56 days.

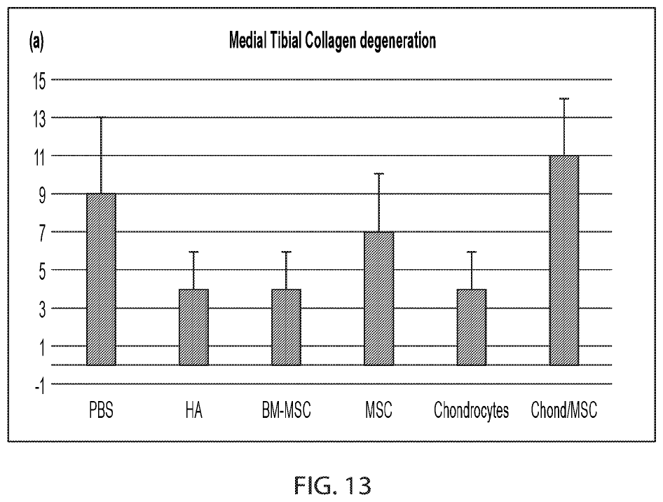

Medial Tibial Collagen Degeneration Scoring: Collagen damage across the medial tibial plateau (most severely affected section of the two halves) was quantified by measuring the total width of the following using an ocular micrometer. Measurements were expressed as a percentage of the total tibial surface width. A reduced percentage indicated reduced collagen damage. FIG. 13 shows measurements made on Study Day 28 and that rats treated with compositions comprising HA, BM-MSC, iPSC-derived MSCs, and iPSC-derived chondrocytes showed reduced collagen damage after treatments.

Femoral Cartilage Degeneration Scoring: General cartilage degeneration includes chondrocyte death/loss, proteoglycan (PG) loss, and collagen loss or fibrillation. The cartilage was divided in to three zones by tissue depth: the outside tissue, middle tissue, and deep tissue so as to study cartilage degeneration across the depth of the tissue. Each zone was scored individually and a sum of all three zones was calculated. Scores were assigned as follows:

0=No degeneration.

0.5=Very minimal degeneration, within a zone less than 5% of the matrix has PG loss mainly with minor chondrocyte loss and little if any collagen matrix loss or damage

1=Minimal degeneration, within a zone 5-10% of the matrix appears non-viable as a result of significant chondrocyte loss (greater than 50% of normal cell density). PG loss is usually present in these areas of cell loss and collagen matrix loss may be present.

2=Mild degeneration, within a zone 11-25% of the matrix appears non-viable as a result of significant chondrocyte loss (greater than 50% of normal cell density). PG loss is usually present in these areas of cell loss and collagen matrix loss may be present.

3=Moderate degeneration, within a zone 26-50% of the matrix appears non-viable as a result of significant chondrocyte loss (greater than 50% of normal cell density). PG loss is usually present in these areas of cell loss and collagen matrix loss may be present.

4=Marked degeneration, within a zone 51-75% of the matrix appears non-viable as a result of significant chondrocyte loss (greater than 50% of normal cell density). PG loss is usually present in these areas of cell loss and collagen matrix loss may be present.

5=Severe degeneration, within a zone 76-100% of the matrix appears non-viable as a result of significant chondrocyte loss (greater than 50% of normal cell density). PG loss is usually present in these areas of cell loss and collagen matrix loss may be present. Lower scores indicate reduced cartilage degeneration.

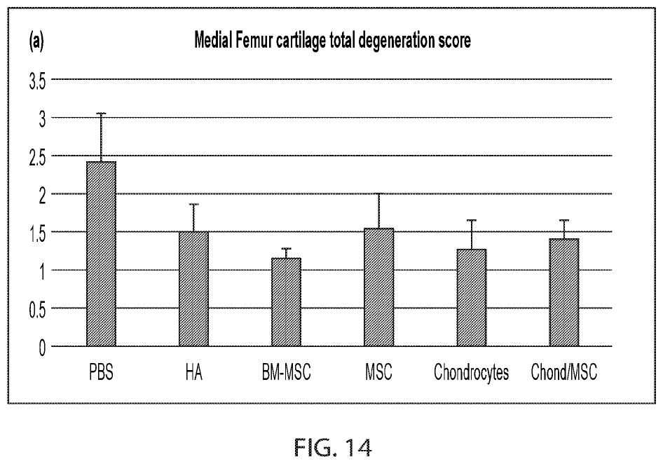

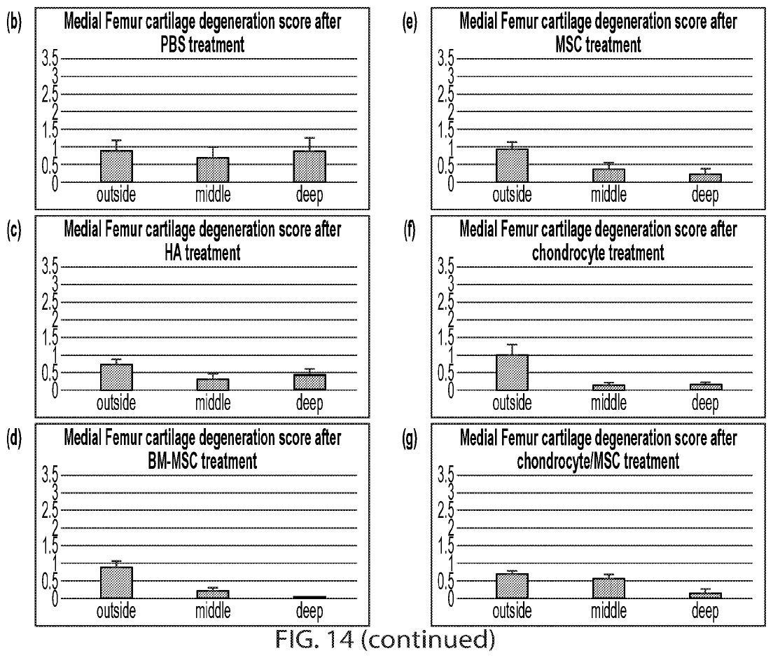

FIG. 14 shows the sum of the scores for all three zones for each of the experimental groups (panel (a)) as well as the individual scores for each zone for each of the experimental groups (panels (b)-(g)). The data indicate that all of the treatments had an effect to reduce the degeneration compared to the PBS control. The cell treatments showed a significant reduction in the cartilage degeneration in the deep layers of the tissue. Furthermore, the treatment with iPSC-derived MSCs and iPSC-derived chondrocytes shows a more even reduction in cartilage damage across all three zones tested.

### Example 7

### Preparation of Chondrons from iPSC-Derived Chondrocytes

Preparatory protocol of iPSC-derived chondrons from iPSC-derived chondrocytes, and protocol for freezing and thawing the iPSC-derived chondrons to produce viable chondrogenic cells was established.

Mature chondrocytes, which are more than 30 days in differentiation and agreccan positive were used for chondrons preparation. Blocks of iPSC-derived chondrocytes (ichondrocyte) were digested using a mixture of 100 U/ml Collagenase II ((Gibco Cat#17101-015), 10U/ml Collagenase P (Roche Cat# 11213857001) in high Glucose-Dulbecco's Modified Eagle Medium (HG-DMEM) at 37° C. The reaction was stopped using ichondrocyte culture media. The pellet formed was then washed twice with HG-DMEM and digestion was assessed using Toluidine Blue staining.

Freezing of chondrons: To freeze chondrons frozen stocks of PRIME-XV FreeezlS (Irvine Scientific) diluted 1:1 with chondrocyte culture media was used. The method of step freezing was used to freeze the chondrons. Specifically, the step freezing protocol comprised the steps of freezing for 20 min at 4° C., followed by freezing for 20 min at −20° C., and then freezing at −80° C. in pre-chilled CoolCell (BioCision) alcohol-free cell freezing container overnight. The chondrons formed were finally stored in liquid nitrogen at −195° C.

Thawing of chondrons: To thaw, the thawing media [5% Human AB serum (Sigma-Aldrich) in DPBS with Ca++/Mg++ (ThermoFisher 14040182)] was brought from 4° C. to room temperature. Just before use, the thawing media was pre-warmed from room temperature to 37° C. 50 ml conical tubes were prepared with 20 ml of pre-warmed thawing media. The cryovials containing frozen chondrons were retrieved from storage and immersed in 37° C. water bath for thawing as rapidly as possible. When the last small ice crystal was still present in the cryovials, the entire content of cryovials was transferred into the previously prepared conical tube containing the pre-warmed thawing media. The tubes were then mixed gently by inversion and the cells were suspended by centrifuge cell suspension at room temperature at 200 × g for 5 min. The supernatant was removed and the cells were re-suspended in 10 ml fresh pre-warmed thawing media. Following this, the cells were centrifuged again at 200 × g for 5 min and re-suspended in fresh pre-warmed thawing media for use.

### Equivalents

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treatment comprising administering a composition to a subject in need thereof, wherein the composition comprises one or more of (i)-(iii) as follows: (i) iPSC-derived Mesenchymal Stem Cells (MSCs), (ii) iPSC-derived chondrocytes, and (iii) iPSC-derived chondrons, wherein the iPSCs from which the one or more of (i)-(iii) were derived have been characterized by HLA typing to determine compatibility for administration to the subject.

2. The method of claim 1, wherein the iPSCs are allogeneic (e.g., from an individual other than the subject).

3. The method of claim 1, wherein the one or more of (i) iPSC-derived Mesenchymal Stem Cells (MSCs), (ii) iPSC-derived chondrocytes, and (iii) iPSC-derived chondrons was retrieved from an indexed-biorepository.

4. The method of claim 1, wherein the composition comprises iPSC-derived MSCs.

5. The method of claim 4, wherein the iPSC-derived MSCs have a transcriptome that comprises transcripts of one or more genes (e.g., at least one, at least two, at least three, at least five, at least seven, at least ten genes) selected from the group consisting of CXCR4, CXCR7, CCL5 (RANTES), IDO 1, A2M, EGFL6, BMP2, BMP4, BMPR1B, IGF2, CILP2, COL2A1.

6. The method of claim 1, wherein the treatment comprises reduction of inflammation.

7. The method of claim 1, wherein the treatment comprises repair of cartilage.

8. The method of claim 1, wherein the composition comprises iPSC-derived MSCs and iPSC-derived chondrocytes.

9. The method of claim 8, wherein a ratio of iPSC-derived MSCs to iPSC-derived chondrocytes is from approximately 0.1:1 to approximately 1:1.

10. The method of claim 8, wherein a ratio of iPSC-derived chondrocytes to iPSC-derived MSCs is from approximately 0.1:1 to approximately 1:1.

11. The method of claim 1, wherein the composition comprises iPSC-derived chondrons.

12. The method of any one of the preceding claims, wherein the administering step comprises administering the composition by injection.

13. The method of any one of the preceding claims, wherein the administering step comprises administering the composition by implantation.

14. The method of any one of the preceding claims, wherein the composition is frozen prior to the administering step.

15. The method of any one of the preceding claims, wherein the composition is thawed prior to the administering step.

16. The method of any one of the preceding claims, comprising storing the composition in an indexed-biorepository prior to the administering step.

17. The method of any one of the preceding claims, wherein the composition was retrieved from an indexed-biorepository prior to the administering step.

18. The method of any one of the preceding claims, wherein the subject is suffering from a disease, a disorder, or an injury that causes cartilage loss and/or damage.

19. The method of any one of the preceding claims, wherein the administering step comprises administering a unit dose of at least approximately 150k iPSC-derived cells.

20. The method of any one of the preceding claims, wherein the composition comprises chondrocytes at a concentration of 3 million cells per mL or greater.

21. The method of any one of the preceding claims, wherein the composition comprises iPSC-derived MSCs and iPSC-derived chondrocytes, wherein the ratio of iPSC-derived MSCs to iPSC-derived chondrocytes in the composition is approximately 1:1.

22. A composition comprising one or more of (i)-(iii) as follows: (i) Induced Pluripotent Stem Cell (iPSC)-derived Mesenchymal Stem Cells (MSCs), (ii) iPSC-derived chondrocytes, and (iii) iPSC-derived chondrons, wherein the iPSCs have been characterized by HLA typing.

23. The composition of claim 22, wherein the composition is injectable.

24. The composition of claim 22, wherein the composition is implantable.

25. The composition of any one of claims 22-24, wherein the composition is frozen.

26. The composition of any one of claims 22-25, wherein the composition is thawed.

27. The composition of any one of claims 22-26, wherein the composition is stored in an indexed-biorepository.

28. The composition of any one of claims 22-27, wherein the composition is retrieved from an indexed-biorepository.

29. The composition of any one of claims 22-28, wherein the one or more of (i) iPSC-derived Mesenchymal Stem Cells (MSCs), (ii) iPSC-derived chondrocytes, and (iii) iPSC-derived chondrons are retrieved from an indexed biorepository.

30. The composition of any one of claims 22-29, wherein the composition comprises iPSC-derived MSCs.

31. The composition of claim 30, wherein the iPSC-derived MSCs have a transcriptome that comprises transcripts of one or more genes (e.g., at least one, at least two, at least three, at least five, at least seven, at least ten genes) selected from the group consisting of CXCR4, CXCR7, CCL5 (RANTES), IDO1, A2M, EGFL6, BMP2, BMP4, BMPR1B, IGF2, CILP2, COL2A1.

32. The composition of any one of claims 22-31, wherein the composition is a unit dose that comprises at least approximately 150k iPSC-derived cells.

33. The composition of any one of claims 22-32, wherein the composition comprises chondrocytes at a concentration of 3 million cells per mL or greater.

34. The composition of any one of claims 22-33, wherein the composition comprises iPSC-derived MSCs and iPSC-derived chondrocytes.

35. The composition of claim 34, wherein a ratio of iPSC-derived MSCs to iPSC-derived chondrocytes in the composition is from approximately 0.1:1 to approximately 1:1.

36. The composition of claim 34, wherein a ratio of iPSC-derived chondrocytes to iPSC-derived MSCs in the composition is from approximately 0.1:1 to approximately 1:1.

37. The composition of claim 34, wherein a ratio of iPSC-derived MSCs to iPSC-derived chondrocytes in the composition is approximately 1:1.

38. The composition of any one of claims 22-37, wherein the composition comprises iPSC-derived chondrons.

39. A method of manufacturing a composition comprising one or more of (I)-(III) as follows: (I) iPSC-derived Mesenchymal Stem Cells (MSCs), (II) iPSC-derived chondrocytes, and (III) iPSC-derived chondrons tailored for treatment of a subject, said method comprising the steps of:
(a) identifying, as compatible with the subject, one or both of (i) and (ii) as follows:
(i) one or more induced pluripotent stem (iPS) cells and/or iPSC-derived cells, said cells being of one or more HLA types each of which is compatible with the subject, and (ii) one or more iPS cell lines and/or one or more iPSC-derived cell lines, said cell lines being of one or more HLA types each of which is compatible with the subject;
(b) retrieving compatible cells corresponding to the one or more cells and/or cell lines identified as compatible with the subject; and
(c) producing the composition using the retrieved compatible cells.

40. The method of claim 39, wherein the compatible cells and/or cells lines are human cells and/or human cell lines.

41. The method of claim 39, wherein the compatible cells and/or cells lines are non-human animal cells and/or non-human animal lines cells.

42. The method of any one of claims 39-41, wherein the compatible cells and/or cell lines are derived from the subject.

43. The method of any one of claims 39-41, wherein the compatible cells and/or cell lines are derived from an individual other than the subject.

44. The method of any one of claims 39-43, wherein the composition comprises iPSC-derived chondrocytes and one or more compatible-cell-secreted species suitable for cartilage repair of the subject, wherein the compatible cell-secreted species are one or more members selected from the group consisting of collagen, proteoglycans, glycosaminoglycans, exosomes, and microvesicles.

45. The method of any one of claims 39-44, wherein the composition comprises iPSC-derived chondrons.

46. The method of any one of claims 39-45, wherein step (c) comprises producing a macroscopic cartilage structure from the retrieved compatible cells or from chondrocytes derived from the retrieved compatible cells.

47. The method of claim 46, wherein step (c) comprises 3D-printing a macroscopic cartilage structure using the composition, wherein the composition is produced from the retrieved compatible cells and/or from chondrocytes derived from the retrieved compatible cells.

48. The method of any one of claims 39-47, wherein step (c) comprises extracting one or more cell-secreted species from the retrieved compatible cells, wherein the cell-secreted species are one or more members selected from the group consisting of collagen, proteoglycans, glycosaminoglycans, exosomes, and microvesicles.

49. The method of any one of claims 39-48, wherein step (b) comprises deriving the compatible cells from a biological sample of the subject.

50. The method of any one of claims 39-49, further comprising (d) freezing the iPSC-derived iPSC-derived Mesenchymal Stem Cells (MSCs), (II) iPSC-derived chondrocytes, and/or (III) iPSC-derived chondrons.

51. The method of any one of claims 39-50, wherein the retrieved compatible cells comprise one or more members selected from the group consisting of iPSCs, MSCs, Retinal Pigment Epithelium (RPEs), chondrocytes, hematopoietic stem cells (HSCs), blood progenitor cells, embryoid bodies, and other iPSC-derived cells.

52. The method of any one of claims 39-51, wherein the subject is human.

53. The method of any one of claims 39-52, wherein step (b) comprises obtaining the compatible cells from a physical repository.

54. The method of any one of claims 39-53, wherein step (b) comprises retrieving the compatible cells using a processor-based query from a user, wherein the query comprises an identification of a cell type indicative of compatibility with the subject.

55. The method of claim 54, wherein the identification of cell type indicative of compatibility with the subject comprises an HLA match.

56. The method of any one of claims 39-55, wherein the composition comprises the retrieved compatible cells.

57. The method of any one of claims 39-56, wherein producing the composition in step (c) comprises exposing the compatible cells to culture and/or differentiation media.

58. The method of claim 57, wherein the composition comprises the compatible cells, the culture media, the differentiation media, and one or more compatible-cell-secreted species, wherein the compatible cell-secreted species are one or more members selected from the group consisting of collagen, proteoglycans, glycosaminoglycans, exosomes, and microvesicles.

59. The method of any one of claims 39-58, comprising dedifferentiating and/or differentiating the one or more iPS-derived cells and/or cell lines identified as compatible with the subject to produce mesenchymal stem cells (MSCs) and/or chondrocytes.

60. The method of claim 59, comprising producing the composition from the MSCs and/or chondrocytes.

61. The method of any one of claims 39-60, wherein the composition is a treatment spray.

62. The method of any one of claims 39-61, wherein the composition is applied topically.

63. The method of any one of claims 39-60, wherein the composition is a treatment injection.

64. The method of any one of claims 39-63, wherein step (b) comprises obtaining the compatible cells from a physical repository, wherein the physical repository comprises an iPS cell line derived from the subject, and wherein step (b) comprises:
storing, by a processor of a computing device, a database comprising a data entry corresponding to each of the iPS cell lines in the physical repository;
receiving, by the processor, a query from a user comprising an identification of the subject; and
matching, by the processor, the query to a data entry of the database, thereby identifying as compatible with the subject the iPS cell line derived from the subject.

65. The method of any one of claims 39-63, wherein step (b) comprises:
storing, by a processor of a computing device, a database comprising a data entry corresponding to each of a plurality of characterized iPS cell and/or iPS cell lines and/or iPSC-derived cell and/or iPSC-derived cell lines or corresponding embryoid bodies, the data entry for each iPS cell and/or iPS cell line and/or iPSC-derived cell and/or iPSC-derived cell line comprising a set of characterized HLA loci corresponding to the iPS cell and/or iPS cell line and/or iPSC-derived cell and/or iPSC-derived cell line;
receiving, by the processor, a query from a user, the query comprising a set of queried HLA loci for the subject; and
retrieving, by the processor, one or more data entries of the database, each representative of an iPS cell and/or cell line and/or an embryoid body and/or an HSC line and/or a blood progenitor line and/or MSC line and/or RPE line and/or chondrocyte line derived from an iPS cell and/or cell line matching the queried HLA loci, thereby identifying cells that match the queried HLA loci for the subject as compatible with the subject.

66. The method of claim 65, wherein the retrieved data entries of the database are exactly matching, partially matching, and/or are identified as compatible with the queried HLA loci.

67. The method of claim 65, wherein the set of characterized HLA loci comprises at least 3 given loci, wherein the given loci are HLA-A, HLA-B, and HLA-DRB.

68. The method of claim 65, wherein the set of characterized HLA loci comprises at least 9 given loci, wherein the given loci are HLA-A, HLA-B, HLA-C, HLA-DRB1, HLA-DRB3, HLA-DRB4, HLA-DRBS, HLA-DQB1, and HLA-DPB1.

69. The method of claim 65, wherein the set of characterized HLA loci comprises at least 3 given loci, wherein the given loci are members selected from the group consisting of HLA-A, HLA-B, HLA-C, HLA-DRB1, HLA-DRB3, HLA-DRB4, HLA-DRBS, HLA-DQB1, and HLA-DPB1.

70. The method of any one of claims 64-69, wherein the queried HLA loci correspond to a subject in need of an HLA matched composition.

71. The method of any one of claims 64-70, wherein the queried HLA loci, is defined by processing and analyzing a sample from a subject in need of an HLA match.

72. The method of any one of claims 64-71, further comprising retrieving characterized cells from the physical repository according to the one or more retrieved data entries matching the queried HLA loci.

73. The method of claim 72, wherein the retrieved characterized cells are one or more members selected from the group consisting of iPS cells, iPS cell lines, embryoid bodies, blood progenitor cells, HSCs, MSCs, RPEs, chondrocytes, iPSC-derived cells, and iPSC-derived cell lines.

74. The method of any one of claims 64-73, further comprising producing MSCs and/or chondrocytes from iPSCs and/or embryoid bodies and/or HSCs and/or blood progenitor cells and/or RPEs of an iPSC line corresponding to the one or more retrieved data entries matching the queried HLA loci.

75. The method of any one of claims 39-74, further comprising administering the composition to the subject.

76. The method of claim 75, wherein the administering step comprises administering the composition to the subject for treatment of a known disease, injury, or condition in the subject, wherein the known disease, injury, or condition is a member selected from the group consisting of rheumatic diseases, cancer, cartilage damage, chondropathy, relapsing polychondritis, osteochondritis dissecans, costochondritis, Chondromalacia patellae, arthritis, and inflammation.

77. A method of any one of claims 64-76, wherein the database comprises a data entry corresponding to each of a plurality of iPS super donor cell lines, the data entry for each super donor cell line comprising a set of characterized HLA loci corresponding to the super donor cell line.

78. The method of claim 77, wherein each of the plurality of iPS super donor cell lines can be used for treatment of a particular subject or particular group of subjects having matching HLA loci with lower risk of immune rejection by the particular subject or particular group of subjects.

79. A treatment comprising a therapeutically effective amount of a composition comprising one or more of (I)-(III) as follows: (I) Induced Pluripotent Stem Cell (iPSC)-derived Mesenchymal Stem Cells (MSCs), (II) iPSC-derived chondrocytes, and (III) iPSC-derived chondrons, for use in a method of treating cartilage loss and/or damage in a subject, wherein the composition is manufactured using one or both of (i) and (ii) as follows: (i) one or more induced pluripotent step (iPS) cells and/or iPSC-derived cells identified as compatible with the subject; and (ii) one or more iPS cell lines and/or one or more iPSC-derived cell lines, wherein the cells and/or cell lines are of one or more HLA types identified as compatible with the subject.

80. The treatment of claim 79, wherein the compatible cells and/or cells lines are human cells and/or human cell lines.

81. The treatment of claim 79, wherein the compatible cells and/or cells lines are non-human animal cells and/or non-human animal lines cells.

82. The treatment of any one of claims 79-81, wherein the compatible cells and/or cell lines are derived from the subject.

83. The treatment of any one of claims 79-81, wherein the compatible cells and/or cell lines are derived from an individual other than the subject.

84. The treatment of any one of claims 79-83, wherein the compatible cells and/or cell lines are identified through the steps of:
determining HLA loci associated with the iPSCs and/or iPS cell lines and/or one or more iPSC-derived cells and/or iPSC-derived cell lines from which the composition is manufactured; and
matching, by a processor of a computing device, the determined HLA loci with the HLA loci of the subject, wherein a match is an exact match or a partial match.

85. The treatment of any one of claims 79-84, wherein the treatment is administered in one or more doses according to a dosing regimen.

86. A method of preparing storable iPSC-derived chondrons from iPSC-derived chondrocytes, the method comprising:
reacting the iPSC-derived chondrocytes (e.g., mature chondrocytes, more than 30 days in differentiation) in digestion media to produce chondrons;
step freezing the chondrons (e.g., by performing a plurality of steps to gradually reduce the temperature in stages prior to introduction to storage in liquid nitrogen); and
storing the step-frozen chondrons.

87. The method of claim 86, further comprising thawing the iPSC-derived chondrons to produce viable chondrogenic cells (e.g., via immersion in a hot (e.g., 37° C.) water bath for thawing as rapidly as possible).

88. The method of claim 87, wherein the thawing is performed in the presence of a pericellular matrix (PCM).

89. The method of any one of claims 86-88, the method comprising retaining a pericellular matrix prior to the step freezing.

90. The method of any one of claims 86-89, wherein the digestion media comprises a collagenase.

91. Use of a composition in the manufacture of a medicament for treatment of a cartilage injury, damage, or defect, wherein the treatment comprises administration of the medicament to a subject in need thereof, wherein the composition comprises one or more of (i)-(iii) as follows:
(i) iPSC-derived Mesenchymal Stem Cells (MSCs), (ii) iPSC-derived chondrocytes, and (iii) iPSC-derived chondrons, wherein the iPSCs have been characterized by HLA typing to determine compatibility for administration to the subject.
